(19) United States
(12) Patent Application Publication
(10) Pub. No.: US 2009/0270450 A1
(43) Pub. Date: Oct. 29, 2009

(54) CHEMICAL COMPOUNDS

(75) Inventors: Leslie Dakin, Waltham, MA (US); Kevin Daly, Waltham, MA (US); David Del Valle, Austin, TX (US); Thomas Gero, Waltham, MA (US); Claude Afona Ogoe, Waltham, MA (US); David Scott, Waltham, MA (US); Xiaolan Zheng, Waltham, MA (US)

Correspondence Address: ASTRAZENECA R&D BOSTON, 35 GATEHOUSE DRIVE, WALTHAM, MA, 02451-1215, US

(73) Assignee: ASTRAZENECA AB, Sodertalje (SE)

(21) Appl. No.: 12/435,856

(22) Filed: May 5, 2009

Related U.S. Application Data
(63) Continuation of application No. PCT/GB2007/004263, filed on Nov. 8, 2007.
(60) Provisional application No. 60/916,182, filed on May 4, 2007.
(60) Provisional application No. 60/865,245, filed on Nov. 10, 2006.

(51) Int. Cl. A61K 31/47; C07D 215/44

(52) U.S. Cl. 514/313; 546/160

(57) ABSTRACT

The invention relates to chemical compounds of formula (I):

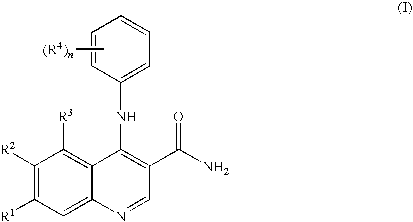

or pharmaceutically acceptable salts thereof which possess CSF-1R kinase inhibitory activity and are accordingly useful for their anti-cancer activity and thus in methods of treatment of the human or animal body. The invention also relates to processes for the manufacture of said chemical compounds, to pharmaceutical compositions containing them and to their use in the manufacture of medicaments of use in the production of an anti-cancer effect in a warm-blooded animal such as man.

The invention relates to chemical compounds, or pharmaceutically acceptable salts thereof, which possess colony stimulating factor 1 receptor (CSF-1R) kinase inhibitory activity and are accordingly useful for their anti-cancer activity and thus in methods of treatment of the human or animal body. The invention also relates to processes for the manufacture of said chemical compounds, to pharmaceutical compositions containing them and to their use in the manufacture of medicaments of use in the production of an anti-cancer effect in a warm-blooded animal such as man.

Receptor tyrosine kinases (RTK's) are a sub-family of protein kinases that play a critical role in cell signalling and are involved in a variety of cancer related processes including cell proliferation, survival, angiogenesis, invasion and metastasis. There are believed to be at least 96 different RTK's including CSF-1R.

CSF-1R or c-fms was originally identified as the oncogene v-fms from the feline sarcoma virus. CSF-1R is a member of the class III RTK's along with c-Kit, fins-related tyrosine kinase 3 (Flt3) and Platelet-derived growth factor receptor α and β (PDGFRα and PDGFRβ). All of these kinases have been implicated in the process of tumorigenesis. CSF-1R is normally expressed as an immature 130 kDa transmembrane protein and ultimately results in a mature 145-160 kDa cell surface N-linked glycosylated protein. Macrophage colony stimulating factor (M-CSF or CSF-1), the ligand for CSF-1R, binds to the receptor resulting in dimerization, auto-phosphorylation of the receptor and subsequent activation of downstream signal transduction cascades (C. J. Sherr, Biochim Biophys Acta, 1988, 948: 225-243).

CSF-1R is normally expressed in myeloid cells of the mononuclear phagocytic lineage and their bone-marrow progenitors as well as the epithelial cells of the ducts and alveoli in the lactating, but not normal resting, breast tissue. CSF-1R activation stimulates the proliferation, survival, motility and differentiation of cells of the monocyte/macrophage lineage. The mature macrophage plays a key role in normal tissue development and immune defence (F. L. Pixley and E. R. Stanley, Trends in Cell Biology, 2004, 14(11): 628-638). For example, osteoblasts secrete CSF-1 and activate the receptor on osteoclastic progenitors resulting in differentiation into mature osteoclasts (S. L. Teitelbaum, Science, 2000, 289: 1504-1508). The CSF-1R axis plays an important role in placental development, embryonic implantation, mammary gland ductal and lobuloalveolar development and lactation (E. Sapi, Exp Biol Med, 2004, 229:1-11).

Transfection of CSF-1R with or without CSF-1 induces transformation and in vivo tumorigenicity of NIH3T3 (Rat2 and ovarian granulosa cells. Autocrine and/or paracrine signaling mechanisms have been implicated in the activation of CSF-1R in the tumour epithelium and tumour associated macrophage. Aberrant expression and activation of CSF-1R and/or its ligand have been found in human myeloid leukaemia, prostate, breast, ovarian, endometrial and a variety of other cancers. A number of studies have demonstrated that the overexpression of CSF-1R is associated with poor prognosis in several of these cancers. In addition, the CSF-1/CSF-1R axis plays a key role in the regulation of tumour-associated macrophage, which have been postulated to play a significant role in tumour angiogenesis, invasion and progression (E. Sapi, Exp Biol Med, 2004, 229:1-11).

Accordingly, the present invention provides a compound of formula (I):

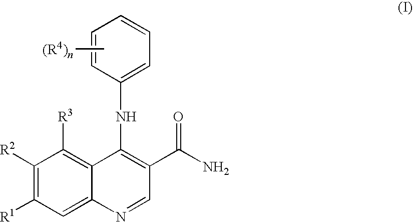

wherein:

one of R¹ and R² is selected from C₁₋₆alkyl, C₂₋₆alkenyl, C₂₋₆alkynyl, carbocyclyl or carbon-linked heterocyclyl; wherein this R¹ or R² may be optionally substituted on carbon by one or more R⁵; and wherein if said heterocyclyl contains an —NH— moiety that nitrogen may be optionally substituted by a group selected from R⁶; and

the other R¹ or R² is selected from hydrogen, halo, nitro, cyano, hydroxy, trifluoromethoxy, amino, carboxy, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, N—(C₁₋₆alkyl)-N—(C₁₋₆alkoxy)amino, C₁₋₆alkanoylamino, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl or carbon-linked heterocyclyl; wherein this R¹ or R² may be optionally substituted on carbon by one or more R⁷; and wherein if said heterocyclyl contains an —NH— moiety that nitrogen may be optionally substituted by a group selected from R⁸;

R³ is hydrogen, or halo;

R⁴ is selected from halo, nitro, cyano, hydroxy, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, N—(C₁₋₆alkyl)-N—(C₁₋₆alkoxy)amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl or heterocyclyl; wherein R⁴ may be optionally substituted on carbon by one or more R⁹; and wherein if said heterocyclyl contains an —NH— moiety that nitrogen may be optionally substituted by a group selected from R¹⁰;

or wherein if two R⁴ groups are on adjacent carbons, they may optionally form a carbocyclic ring or a heterocyclic ring; wherein said carbocyclic ring or heterocyclic ring may be optionally substituted on carbon by one or more R¹¹; and wherein if said heterocyclic ring contains an —NH— moiety that nitrogen may be optionally substituted by a group selected from R¹²;

n is 0-3; wherein the values of R⁴ are the same or different;

R⁵, R⁷, R⁹ and R¹¹ are independently selected from halo, nitro, cyano, hydroxy, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, N—(C₁₋₆alkyl)-N—(C₁₋₆alkoxy)amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, C₁₋₆alkoxycarbonylamino, N—(C₁₋₆alkyl)sulphamoyl, N,N—(C₁₋₆alkyl)₂sulphamoyl, C₁₋₆alkylsulphonylamino, carbocyclyl-R¹³— or heterocyclyl-R¹⁴—; wherein R⁵, R⁷, R⁹ and R¹¹ independently of each other may be optionally substituted on carbon by one or more R¹⁵; and wherein if said heterocyclyl contains an —NH— moiety that nitrogen may be optionally substituted by a group selected from R¹⁶;

R¹³ and R¹⁴ are independently selected from a direct bond, —O—, —N(R¹⁷)—, —C(O)—, —N(R¹⁸)C(O)—, —C(O)N(R¹⁹)—, —S(O)ₛ—, —SO₂N(R²⁰)— or —N(R²¹)SO₂—; wherein R¹⁷, R¹⁸, R¹⁹, R²⁰ and R²¹ are independently selected from hydrogen or C₁₋₆alkyl and s is 0-2;

R⁶, R⁸, R¹⁰, R¹² and R¹⁶ are independently selected from C₁₋₆alkyl, C₁₋₆alkanoyl, C₁₋₆alkylsulphonyl, C₁₋₆alkoxycarbonyl, carbamoyl, N—(C₁₋₆alkyl)carbamoyl, N,N—(C₁₋₆alkyl)carbamoyl, benzyl, benzyloxycarbonyl, benzoyl and phenylsulphonyl; wherein R⁶, R⁸, R¹⁰, R¹² and R¹⁶ independently of each other may be optionally substituted on carbon by one or more R²²; and

R¹⁵ and R²² are independently selected from halo, nitro, cyano, hydroxy, trifluoromethoxy, trifluoromethyl, amino, carboxy, carbamoyl, mercapto, sulphamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, phenyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl or N-methyl-N-ethylsulphamoyl; or a pharmaceutically acceptable salt thereof;

with the proviso that if R¹ is phenyl or pyrid-4-yl, R² is not hydrogen.

According to a further feature of the invention there is provided a compound of formula (I) (as depicted above) wherein:

one of R¹ and R² is selected from C₁₋₆alkyl, C₂₋₆alkenyl, C₂₋₆alkynyl, carbocyclyl or carbon-linked heterocyclyl; wherein this R¹ or R² may be optionally substituted on carbon by one or more R⁵; and wherein if said heterocyclyl contains an —NH— moiety that nitrogen may be optionally substituted by a group selected from R⁶; and

the other R¹ or R² is selected from hydrogen, halo, nitro, cyano, hydroxy, trifluoromethoxy, amino, carboxy, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, N—(C₁₋₆alkyl)-N—(C₁₋₆alkoxy)amino, C₁₋₆alkanoylamino, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl or carbon-linked heterocyclyl; wherein this R¹ or R² may be optionally substituted on carbon by one or more R⁷; and wherein if said heterocyclyl contains an —NH— moiety that nitrogen may be optionally substituted by a group selected from R⁸;

R³ is hydrogen, or halo;

R⁴ is selected from halo, nitro, cyano, hydroxy, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, N—(C₁₋₆alkyl)-N—(C₁₋₆alkoxy)amino, C₁₋₁₆alkanoylamino, N—(C₁₋₆alkyl)carbamoyl, N,N—(C₁₋₆alkyl)₂-carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl or heterocyclyl; wherein R⁴ may be optionally substituted on carbon by one or more R⁹; and wherein if said heterocyclyl contains an —NH— moiety that nitrogen may be optionally substituted by a group selected from R¹⁰;

or wherein if two R⁴ groups are on adjacent carbons, they may optionally form a carbocyclic ring or a heterocyclic ring; wherein said carbocyclic ring or heterocyclic ring may be optionally substituted on carbon by one or more R¹¹; and wherein if said heterocyclic ring contains an —NH— moiety that nitrogen may be optionally substituted by a group selected from R¹²

n is 0-3; wherein the values of R⁴ are the same or different;

R⁵, R⁷, R⁹ and R¹¹ are independently selected from halo, nitro, cyano, hydroxy, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, N—(C₁₋₆alkyl)-N—(C₁₋₆alkoxy)amino, C₁₋₆alkanoylamino, N—(C₁₋₆alkyl)carbamoyl, N,N—(C₁₋₆alkyl)₂-carbamoyl, C₁₋₆alkylS(O)ₐ wherein a is 0 to 2, C₁₋₆alkoxycarbonyl, C₁₋₆alkoxycarbonylamino, N—(C₁₋₆alkyl)sulphamoyl, N,N—(C₁₋₆alkyl)₂sulphamoyl, C₁₋₆alkylsulphonylamino, carbocyclyl-R¹³— or heterocyclyl-R¹⁴—; wherein R⁵, R⁷, R⁹ and R¹¹ independently of each other may be optionally substituted on carbon by one or more R¹⁵; and wherein if said heterocyclyl contains an —NH— moiety that nitrogen may be optionally substituted by a group selected from R¹⁶;

R¹³ and R¹⁴ are independently selected from a direct bond, —O—, —N(R¹⁷)—, —C(O)—, —N(R¹⁸)C(O)—, —C(O)N(R¹⁹)—, —S(O)ₛ—, —SO₂N(R²⁰)— or —N(R²¹)SO₂—; wherein R¹⁷, R¹⁸, R¹⁹, R²⁰ and R²¹ are independently selected from hydrogen or C₁₋₆alkyl and s is 0-2;

R⁶, R⁸, R¹⁰, R¹² and R¹⁶ are independently selected from C₁₋₆alkyl, C₁₋₆alkanoyl, C₁₋₆alkylsulphonyl, C₁₋₆alkoxycarbonyl, carbamoyl, N—(C₁₋₆alkyl)carbamoyl, N,N—(C₁₋₆alkyl)carbamoyl, benzyl, benzyloxycarbonyl, benzoyl and phenylsulphonyl; and

R¹⁵ is selected from halo, nitro, cyano, hydroxy, trifluoromethoxy, trifluoromethyl, amino, carboxy, carbamoyl, mercapto, sulphamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, phenyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl or N-methyl-N-ethylsulphamoyl;

or a pharmaceutically acceptable salt thereof;
with the proviso that if R¹ is phenyl or pyrid-4-yl, R² is not hydrogen.

In this specification the term “alkyl” includes both straight and branched chain alkyl groups. References to individual alkyl groups such as “propyl” are specific for the straight chain version only and references to individual branched chain alkyl groups such as ‘isopropyl’ are specific for the branched chain version only. For example, “C₁₋₆alkyl” includes C₁₋₄alkyl, C₁₋₃alkyl, propyl, isopropyl and t-butyl. A similar convention applies to other radicals, for example “phenylC₁₋₆alkyl” includes phenylC₁₋₄alkyl, benzyl, 1-phenylethyl and 2-phenylethyl. The term “halo” refers to fluoro, chloro, bromo and iodo.

Where optional substituents are chosen from “one or more” groups it is to be understood that this definition includes all substituents being chosen from one of the specified groups or the substituents being chosen from two or more of the specified groups.

A “heterocyclyl” is a saturated, partially saturated or unsaturated, mono or bicyclic ring containing 4-12 atoms of which at least one atom is chosen from nitrogen, sulphur or oxygen, which may, unless otherwise specified, be carbon or nitrogen linked, wherein a —CH₂-group can optionally be replaced by a —C(O)— and a ring sulphur atom may be optionally oxidised to form the S-oxides. Examples and suitable values of the term “heterocyclyl” are morpholino, piperidyl, pyridyl, pyranyl, pyrrolyl, pyrazolyl, isothiazolyl, indolyl, quinolyl, thienyl, 1,3-benzodioxolyl, thiadiazolyl, piperazinyl, thiazolidinyl, pyrrolidinyl, thiomorpholino, pyrrolinyl, homopiperazinyl, 3,5-dioxapiperidinyl, tetrahydropyranyl, imidazolyl, pyrimidyl, pyrazinyl, pyridazinyl, isoxazolyl, N-methylpyrrolyl, 4-pyridone, 1-isoquinolone, 2-pyrrolidone, 4-thiazolidone, pyridine-N-oxide and quinoline-N-oxide. A particular example of the term “heterocyclyl” is pyrazolyl. In one aspect of the invention a “heterocyclyl” is a saturated, partially saturated or unsaturated, monocyclic ring containing 5 or 6 atoms of which at least one atom is chosen from nitrogen, sulphur or oxygen, it may, unless otherwise specified, be carbon or nitrogen linked, a —CH₂— group can optionally be replaced by a —C(O)— and a ring sulphur atom may be optionally oxidised to form the S-oxides.

A “carbocyclyl” is a saturated, partially saturated or unsaturated, mono or bicyclic carbon ring that contains 3-12 atoms; wherein a —CH₂— group can optionally be replaced by a —C(O)—. Particularly “carbocyclyl” is a monocyclic ring containing 5 or 6 atoms or a bicyclic ring containing 9 or 10 atoms. Suitable values for “carbocyclyl” include cyclopropyl, cyclobutyl, 1-oxocyclopentyl, cyclopentyl, cyclopentenyl, cyclohexyl, cyclohexenyl, phenyl, naphthyl, tetralinyl, indanyl or 1-oxoindanyl. A particular example of “carbocyclyl” is phenyl.

“If two R⁴ groups are on adjacent carbons, they may optionally form a carbocyclic ring or a heterocyclic ring”. Said “carbocyclic ring” or a “heterocyclic ring” is therefore fused to the phenyl ring of formula (I).

A “carbocyclic ring” is a partially saturated or totally unsaturated, monocyclic ring that contains 3-8 carbon atoms of which two are shared with the phenyl ring in formula (I); wherein a —CH₂— group can optionally be replaced by a —C(O)—. Suitable examples of a “carbocyclic ring” fused to the phenyl ring in formula (I) include indanyl (carbocyclic ring is a partially saturated 5 membered ring) and naphthyl (carbocyclic ring is a totally unsaturated 6 membered ring).

A “heterocyclic ring” is a partially saturated or totally unsaturated, monocyclic ring containing 4-8 atoms of which at least one atom is chosen from nitrogen, sulphur or oxygen and two atoms are carbon atoms shared with the phenyl ring in formula (I); wherein a —CH₂-group can optionally be replaced by a —C(O)— and a ring sulphur atom may be optionally oxidised to form the S-oxides. Suitable examples of a “heterocyclic ring” fused to the phenyl ring in formula (I) include indolinyl (heterocyclic ring is a partially saturated 5 membered ring containing one nitrogen atom) and quinoxalinyl (heterocyclic ring is a totally unsaturated 6 membered ring containing two nitrogen atoms).

An example of “C₁₋₆alkanoyloxy” is acetoxy. Examples of “C₁₋₆alkoxycarbonyl” include methoxycarbonyl, ethoxycarbonyl, n- and t-butoxycarbonyl. Examples of “C₁₋₆alkoxy” include methoxy, ethoxy and propoxy. Examples of “C₁₋₆alkanoylamino” include formamido, acetamido and propionylamino. Examples of “C₁₋₆alkylS(O)ₐ wherein a is 0 to 2” include methylthio, ethylthio, methylsulphinyl, ethylsulphinyl, mesyl and ethylsulphonyl. Examples of “C₁₋₆alkanoyl” include propionyl and acetyl. Examples of “N—(C₁₋₆alkyl)amino” include methylamino and ethylamino. Examples of “N,N—(C₁₋₆alkyl)₂-amino” include di-N-methylamino, di-(N-ethyl)amino and N-ethyl-N-methylamino. Examples of “C₂₋₆alkenyl” are vinyl, allyl and 1-propenyl. Examples of “C₂₋₆alkynyl” are ethynyl, 1-propynyl and 2-propynyl. Examples of “N—(C₁₋₆alkyl)sulphamoyl” are N-(methyl)sulphamoyl and N-(ethyl)sulphamoyl. Examples of “N—(C₁₋₆alkyl)₂sulphamoyl” are N,N-(dimethyl)sulphamoyl and N-(methyl)-N-(ethyl)sulphamoyl. Examples of “N—(C₁₋₆alkyl)carbamoyl” are N—(C₁₋₄alkyl)carbamoyl, methylaminocarbonyl and ethylaminocarbonyl. Examples of “N,N—(C₁₋₆alkyl)₂-carbamoyl” are N,N—(C₁₋₄alkyl)₂-carbamoyl, dimethylaminocarbonyl and methylethylaminocarbonyl. Examples of “C₁₋₆alkylsulphonyl” are mesyl, ethylsulphonyl and isopropylsulphonyl. Examples Of “C₁₋₆alkylsulphonylamino” are mesylamino, ethylsulphonylamino and isopropylsulphonylamino. Examples of “C₁₋₆alkoxycarbonylamino” are methoxycarbonylamino and t-butoxycarbonylamino. Examples of “C₁₋₆alkoxycarbonylamino” include methoxycarbonylamino and t-butoxycarbonylamino.

A suitable pharmaceutically acceptable salt of a compound of the invention is, for example, an acid-addition salt of a compound of the invention which is sufficiently basic, for example, an acid-addition salt with, for example, an inorganic or organic acid, for example hydrochloric, hydrobromic, sulphuric, phosphoric, trifluoroacetic, citric or maleic acid. In addition a suitable pharmaceutically acceptable salt of a compound of the invention which is sufficiently acidic is an alkali metal salt, for example a sodium or potassium salt, an alkaline earth metal salt, for example a calcium or magnesium salt, an ammonium salt or a salt with an organic base which affords a physiologically-acceptable cation, for example a salt with methylamine, dimethylamine, trimethylamine, piperidine, morpholine or tris-(2-hydroxyethyl)amine.

Some compounds of the formula (I) may have chiral centres and/or geometric isomeric centres (E- and Z-isomers), and it is to be understood that the invention encompasses all such optical, diastereoisomers and geometric isomers that possess CSF-1R kinase inhibitory activity. The invention further relates to any and all tautomeric forms of the compounds of the formula (I) that possess CSF-1R kinase inhibitory activity.

It is also to be understood that certain compounds of the formula (I) can exist in solvated as well as unsolvated forms such as, for example, hydrated forms. It is to be understood that the invention encompasses all such solvated forms which possess CSF-1R kinase inhibitory activity.

Particular values of variable groups are as follows. Such values may be used where appropriate with any of the definitions, claims or embodiments defined hereinbefore or hereinafter.

R¹ is selected from C₁₋₆alkyl, C₂₋₆alkenyl, C₂₋₆alkynyl, carbocyclyl or carbon-linked heterocyclyl; wherein R¹ may be optionally substituted on carbon by one or more R⁵; and wherein if said heterocyclyl contains an —NH— moiety that nitrogen may be optionally substituted by a group selected from R⁶.

R¹ is selected from C₁₋₆alkyl, C₂₋₆alkenyl or C₂₋₆alkynyl; wherein R¹ may be optionally substituted on carbon by one or more R⁵.

R² is selected from C₁₋₆alkyl, C₂₋₆alkenyl, C₂₋₆alkynyl, carbocyclyl or carbon-linked heterocyclyl; wherein R² may be optionally substituted on carbon by one or more R⁵; and wherein if said heterocyclyl contains an —NH— moiety that nitrogen may be optionally substituted by a group selected from R⁶.

R² is selected from C₁₋₆alkyl, C₂₋₆alkenyl or C₂₋₆alkynyl; wherein R² may be optionally substituted on carbon by one or more R⁵

R¹ is selected from hydrogen, halo, nitro, cyano, hydroxy, trifluoromethoxy, amino, carboxy, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, N—(C₁₋₆alkyl)-N—(C₁₋₆alkoxy)amino, C₁₋₆alkanoylamino, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl or carbon-linked heterocyclyl; wherein R¹ may be optionally substituted on carbon by one or more R⁷; and wherein if said heterocyclyl contains an —NH— moiety that nitrogen may be optionally substituted by a group selected from R⁸.

R¹ is selected from C₁₋₆alkoxy.

R¹ is selected from methoxy.

R¹ is selected from ethoxy.

R¹ is carbocyclyl or C₁₋₆alkoxy.

R¹ is cyclopropyl, methoxy or ethoxy. R² is selected from hydrogen, halo, nitro, cyano, hydroxy, trifluoromethoxy, amino, carboxy, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, N—(C₁₋₆alkyl)-N—(C₁₋₆alkoxy)amino, C₁₋₆alkanoylamino, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl or carbon-linked heterocyclyl; wherein R² may be optionally substituted on carbon by one or more R⁷; and wherein if said heterocyclyl contains an —NH— moiety that nitrogen may be optionally substituted by a group selected from R⁸.

R² is selected from C₁₋₆alkoxy.

R² is selected from methoxy.

R² is selected from ethoxy.

R² is selected from C₁₋₆alkyl, C₂₋₆alkynyl, carbocyclyl or carbon-linked heterocyclyl; wherein this R² may be optionally substituted on carbon by one or more R⁵; and wherein if said heterocyclyl contains an —NH— moiety that nitrogen may be optionally substituted by a group selected from R⁶; or R² is selected from C₁₋₆alkoxy; wherein

R⁵ is selected from hydroxy, amino, C₁₋₆alkyl, C₁₋₆alkoxy, N,N—(C₁₋₆alkyl)₂amino, C₁₋₆alkoxycarbonylamino, carbocyclyl-R¹³— or heterocyclyl-R¹⁴—;

R¹³ and R¹⁴ are independently selected from a direct bond, —O—, —N(R¹⁷)—; wherein R¹⁷ is hydrogen;

R⁶ is selected from C₁₋₆alkyl, C₁₋₆alkanoyl, C₁₋₆alkoxycarbonyl; wherein R⁶ may be optionally substituted on carbon by one or more R²²; and

R²² is selected from hydroxy or methoxy.

R² is selected from propyl, prop-1-ynyl, cyclopropyl, isoxazol-4-yl, pyrrol-2-yl, pyrimidin-5-yl, pyrid-4-yl, pyrazol-4-yl, 1,2,3,6-tetrahydropyrid-4-yl, piperidin-4-yl or pyrid-3-yl; wherein this R² may be optionally substituted on carbon by one or more R⁵; and wherein if said heterocyclyl contains an —NH— moiety that nitrogen may be optionally substituted by a group selected from R⁶; or R² is selected from methoxy;

R⁵ is selected from hydroxy, amino, methyl, methoxy, dimethylamino, t-butoxycarbonylamino, cyclopropyl-R¹³—, tetrahydro-2H-pyran-2-yl-R¹⁴— or piperidin-1-yl-R¹⁴—;

R¹³ and R¹⁴ are independently selected from a direct bond, —O—, —N(R¹⁷)—; wherein R¹⁷ is hydrogen;

R⁶ is selected from methyl, ethyl, isopropyl, t-butyl, acetyl, propionyl, t-butoxycarbonyl; wherein R⁶ may be optionally substituted on carbon by one or more R²²; and

R²² is selected from hydroxy or methoxy.

R² is selected from 1-(2-hydroxyethyl)-4-piperidyl, 1-(3-methoxypropanoyl)-4-piperidyl, 1,2,3,6-tetrahydropyridin-4-yl, 1-[(2R)-2-hydroxypropanoyl]-4-piperidyl, 1-acetyl-3,6-dihydro-2H-pyridin-4-yl, 1-acetyl-4-piperidyl, 1H-pyrazol-4-yl, 1H-pyrrol-2-yl, 1-isobutylpyrazol-4-yl, 1-isopropyl-4-piperidyl, 1-methyl-4-piperidyl, 1-tert-butoxycarbonyl-3,6-dihydro-2H-pyridin-4-yl, 3-(1-piperidyl)propyl, 3-(cyclopropylamino)propyl, 3,5-dimethylisoxazol-4-yl, 3-aminopropyl, 3-dimethylaminopropyl, 3-hydroxyprop-1-ynyl, 3-hydroxypropyl, 3-pyridyl, 4-piperidyl, 4-pyridyl, 6-methoxy-3-pyridyl, 6-oxo-1H-pyridin-3-yl, cyclopropyl, methoxy, pyrimidin-5-yl, 3-(t-butoxycarbonylamino)propyl or 3-(tetrahydro-2H-pyran-2-yloxy)propyl.

One of R¹ and R² is selected from C₁₋₆alkyl, C₂₋₆alkynyl, carbocyclyl or carbon-linked heterocyclyl; wherein this R¹ or R² may be optionally substituted on carbon by one or more R⁵; and wherein if said heterocyclyl contains an —NH— moiety that nitrogen may be optionally substituted by a group selected from R⁶; and

the other R¹ or R² is selected from C₁₋₆alkoxy;

R⁵ is selected from hydroxy, amino, C₁₋₆alkyl, C₁₋₆alkoxy, N,N—(C₁₋₆alkyl)₂amino, C₁₋₆alkoxycarbonylamino, carbocyclyl-R¹³— or heterocyclyl-R¹⁴—;

R¹³ and R¹⁴ are independently selected from a direct bond, —O—, —N(R¹⁷)—; wherein R¹⁷ is hydrogen;

R⁶ is selected from C₁₋₆alkyl, C₁₋₆alkanoyl, C₁₋₆alkoxycarbonyl; wherein R⁶ may be optionally substituted on carbon by one or more R²²; and

R²² is selected from hydroxy or methoxy.

One of R¹ and R² is selected from C₁₋₆alkyl, C₂₋₆alkynyl, carbocyclyl or carbon-linked heterocyclyl; wherein this R¹ or R² may be optionally substituted on carbon by one or more R⁵; and wherein if said heterocyclyl contains an —NH— moiety that nitrogen may be optionally substituted by a group selected from R⁶; and

the other R¹ or R² is selected from C₁₋₆alkoxy; wherein

R⁵ is selected from hydroxy, amino, C₁₋₆alkyl, C₁₋₆alkoxy, N,N—(C₁₋₆alkyl)₂amino, C₁₋₆alkoxycarbonylamino, carbocyclyl-R¹³— or heterocyclyl-R¹⁴—;

R¹³ and R¹⁴ are independently selected from a direct bond, —O—, or —N(R¹⁷)—; wherein R¹⁷ is hydrogen;

R⁶ is selected from C₁₋₆alkyl, C₁₋₆alkanoyl and C₁₋₆alkoxycarbonyl.

One of R¹ and R² is selected from C₁₋₆alkyl, C₂₋₆alkynyl, carbocyclyl or carbon-linked heterocyclyl; wherein this R¹ or R² may be optionally substituted on carbon by one or more R⁵; and wherein if said heterocyclyl contains an —NH— moiety that nitrogen may be optionally substituted by a group selected from R⁶; and

the other R¹ or R² is selected from C₁₋₆alkoxy; wherein

R⁵ is selected from hydroxy, amino, C₁₋₆alkyl, N,N—(C₁₋₆alkyl)₂amino, C₁₋₆alkoxycarbonylamino, carbocyclyl-R¹³— or heterocyclyl-R¹⁴—;

R¹³ and R¹⁴ are independently selected from a direct bond, —O— or —N(R¹⁷)—; wherein R¹⁷ is selected from hydrogen; and

R⁶ is selected from C₁₋₆alkyl or C₁₋₆alkoxycarbonyl.

One of R¹ and R² is selected from propyl, prop-1-ynyl, cyclopropyl, isoxazol-4-yl, pyrrol-2-yl, pyrimidin-5-yl, pyrid-4-yl, pyrazol-4-yl, 1,2,3,6-tetrahydropyrid-4-yl, piperidin-4-yl or pyrid-3-yl; wherein this R¹ or R² may be optionally substituted on carbon by one or more R⁵; and wherein if said heterocyclyl contains an —NH— moiety that nitrogen may be optionally substituted by a group selected from R⁶; and

the other R¹ or R² is selected from methoxy or ethoxy;

R⁵ is selected from hydroxy, amino, methyl, methoxy, dimethylamino, t-butoxycarbonylamino, cyclopropyl-R¹³—, tetrahydro-2H-pyran-2-yl-R¹⁴— or piperidin-1-yl-R¹⁴—;

R¹³ and R¹⁴ are independently selected from a direct bond, —O—, —N(R¹⁷)—; wherein R¹⁷ is hydrogen;

R⁶ is selected from methyl, ethyl, isopropyl, t-butyl, acetyl, propionyl, t-butoxycarbonyl; wherein R⁶ may be optionally substituted on carbon by one or more R²²; and

R²² is selected from hydroxy or methoxy.

One of R¹ and R² is selected from propyl, prop-1-ynyl, cyclopropyl, 1,2,3,6-tetrahydropyridin-4-yl, isoxazol-4-yl, pyrazol-4-yl, 6-oxo-1H-pyridin-3-yl, 3-pyridyl, pyrrol-2-yl, 4-piperidyl, 4-pyridyl, pyrimidin-5-yl, pyrazolyl-4-yl or 3,6-dihydro-2H-pyridin-4-yl; wherein this R¹ or R² may be optionally substituted on carbon by one or more R⁵; and wherein if said heterocyclyl contains an —NH— moiety that nitrogen may be optionally substituted by a group selected from R⁶; and

the other R¹ or R² is selected from methoxy or ethoxy; wherein

R⁵ is selected from hydroxy, amino, methyl, methoxy, dimethylamino, t-butoxycarbonylamino, cyclopropyl-R¹³—, tetrahydropyran-2-yl-R¹⁴— or piperid-1-yl-R¹⁴—;

R¹³ and R¹⁴ are independently selected from a direct bond, —O—, or —N(R¹⁷)—; wherein R¹⁷ is hydrogen;

R⁶ is selected from methyl, isopropyl, isobutyl, acetyl and t-butoxycarbonyl.

One of R¹ and R² is selected from propyl, prop-1-ynyl, cyclopropyl, isoxazol-4-yl, pyrrol-2-yl, pyrimidin-5-yl, pyridin-3-yl, pyrazol-4-yl, 1,2,3,6-tetrahydropyridin-4-yl or pyridin-4-yl; wherein this R¹ or R² may be optionally substituted on carbon by one or more R⁵; and wherein if said heterocyclyl contains an —NH— moiety that nitrogen may be optionally substituted by a group selected from R⁶; and

the other R¹ or R² is selected from methoxy or ethoxy; wherein

R⁵ is selected from hydroxy, amino, C₁₋₆alkyl, dimethylamino, t-butoxycarbonylamino, cyclopropyl-R¹³— or piperidin-1-yl-R¹⁴—;

R¹³ and R¹⁴ are independently selected from a direct bond, —O— or —N(R¹⁷)—; wherein R¹⁷ is selected from hydrogen; and

R⁶ is selected from C₁₋₆alkyl or C₁₋₆alkoxycarbonyl.

One of R¹ and R² is selected from 3-hydroxypropyl, 3-piperidin-1-ylpropyl, 3-(cyclopropylamino)propyl, 3-dimethylaminopropyl, 3-aminopropyl, 3-(t-butoxycarbonylamino)propyl, 3-(3,4,5,6-tetrahydropyran-2-yloxy)propyl, cyclopropyl, 3-hydroxyprop-1-ynyl, pyridin-3-yl, 3,5-dimethylisoxazol-4-yl, pyrrol-2-yl, pyrimidin-5-yl, pyridin-4-yl, pyrazol-4-yl, 1-(t-butoxycarbonyl)-1,2,3,6-tetrahydropyridin-4-yl and 1-isobutylpyrazol-4-yl; and

the other R¹ or R² is selected from methoxy or ethoxy.

One of R¹ and R² is selected from 1-(2-hydroxyethyl)-4-piperidyl, 1-(3-methoxypropanoyl)-4-piperidyl, 1,2,3,6-tetrahydropyridin-4-yl, 1-[(2R)-2-hydroxypropanoyl]-4-piperidyl, 1-acetyl-3,6-dihydro-2H-pyridin-4-yl, 1-acetyl-4-piperidyl, 1H-pyrazol-4-yl, 1H-pyrrol-2-yl, 1-isobutylpyrazol-4-yl, 1-isopropyl-4-piperidyl, 1-methyl-4-piperidyl, 1-tert-butoxycarbonyl-3,6-dihydro-2H-pyridin-4-yl, 3-(1-piperidyl)propyl, 3-(cyclopropylamino)propyl, 3,5-dimethylisoxazol-4-yl, 3-aminopropyl, 3-dimethylaminopropyl, 3-hydroxyprop-1-ynyl, 3-hydroxypropyl, 3-pyridyl, 4-piperidyl, 4-pyridyl, 6-methoxy-3-pyridyl, 6-oxo-1H-pyridin-3-yl, cyclopropyl, pyrimidin-5-yl, 3-(t-butoxycarbonylamino)propyl or 3-(tetrahydro-2H-pyran-2-yloxy)propyl;

the other R¹ or R² is selected from methoxy or ethoxy.

One of R¹ and R² is selected from 1,2,3,6-tetrahydropyridin-4-yl, 1-acetyl-3,6-dihydro-2H-pyridin-4-yl, 1H-pyrazol-4-yl, 1H-pyrrol-2-yl, 1-isobutylpyrazol-4-yl, 1-isopropyl-4-piperidyl, 1-methyl-4-piperidyl, 1-tert-butoxycarbonyl-3,6-dihydro-2H-pyridin-4-yl, 3-(1-piperidyl)propyl, 3-(cyclopropylamino)propyl, 3,5-dimethylisoxazol-4-yl, 3-aminopropyl, 3-dimethylaminopropyl, 3-hydroxyprop-1-ynyl, 3-hydroxypropyl, 3-pyridyl, 4-piperidyl, 4-pyridyl, 6-methoxy-3-pyridyl, 6-oxo-1H-pyridin-3-yl, cyclopropyl and pyrimidin-5-yl;

the other R¹ or R² is selected from methoxy or ethoxy.

R¹ is methoxy, ethoxy or cyclopropyl.

R² is 3-(t-butoxycarbonylamino)propyl, 3-(3,4,5,6-tetrahydropyran-2-yloxy)propyl, 1-acetyl-3,6-dihydro-2H-pyridin-4-yl, 1-isobutylpyrazol-4-yl, 1-isopropyl-4-piperidyl, 1,2,3,6-tetrahydropyridin-4-yl, 1H-pyrazol-4-yl, 1H-pyrrol-2-yl, 1-methyl-4-piperidyl, 1-tert-butoxycarbonyl-3,6-dihydro-2H-pyridin-4-yl, 3-(1-piperidyl)propyl, 3-(cyclopropylamino)propyl, 3,5-dimethylisoxazol-4-yl, 3-aminopropyl, 3-dimethylaminopropyl, 3-hydroxyprop-1-ynyl, 3-hydroxypropyl, 3-pyridyl, 4-piperidyl, 4-pyridyl, 6-methoxy-3-pyridyl, 6-oxo-1H-pyridin-3-yl, cyclopropyl, methoxy or pyrimidin-5-yl.

R³ is hydrogen.

R³ is halo.

R⁴ is selected from halo and C₁₋₆alkyl.

R⁴ is selected from fluoro, chloro, methyl and ethyl.

R⁴ is selected from fluoro, chloro and ethyl.

n is 0.

n is 1.

n is 2; wherein the values of R⁴ are the same or different.

n is 3; wherein the values of R⁴ are the same or different.

n is 1 or 2; wherein the values of R⁴ are the same or different.

R⁴, n and the phenyl ring to which they are attached form 2,3-dichlorophenyl, 2,4-difluorophenyl, 2-fluoro-4-methyl-phenyl, 2-fluoro-5-methyl-phenyl, 3,4-dichlorophenyl, 3-chloro-2-fluoro-phenyl, 3-chloro-4-fluoro-phenyl or 4-ethylphenyl.

Therefore in a further aspect of the invention there is provided a compound of formula (I) (as depicted above) wherein:

one of R¹ and R² is selected from C₁₋₆alkyl, C₂₋₆alkynyl, carbocyclyl or carbon-linked heterocyclyl; wherein this R¹ or R² may be optionally substituted on carbon by one or more R⁵; and wherein if said heterocyclyl contains an —NH— moiety that nitrogen may be optionally substituted by a group selected from R⁶; and

the other R¹ or R² is selected from C₁₋₆alkoxy;

R³ is hydrogen;

R⁴ is selected from halo and C₁₋₆alkyl;

n is 1 or 2; wherein the values of R⁴ are the same or different;

R⁵ is selected from hydroxy, amino, C₁₋₆alkyl, N,N—(C₁₋₆alkyl)₂amino, C₁₋₆alkoxycarbonylamino, carbocyclyl-R¹³— or heterocyclyl-R¹⁴—;

R⁶ is selected from C₁₋₆alkyl or C₁₋₆alkoxycarbonyl; and

R¹³ and R¹⁴ are independently selected from a direct bond, —O— or —N(R¹⁷)—; wherein R¹⁷ is selected from hydrogen;

or a pharmaceutically acceptable salt thereof.

Therefore in a further aspect of the invention there is provided a compound of formula (I) (as depicted above) wherein:

one of R¹ and R² is selected from C₁₋₆alkyl, C₂₋₆alkynyl, carbocyclyl or carbon-linked heterocyclyl; wherein this R¹ or R² may be optionally substituted on carbon by one or more R⁵; and wherein if said heterocyclyl contains an —NH— moiety that nitrogen may be optionally substituted by a group selected from R⁶; and

the other R¹ or R² is selected from C₁₋₆alkoxy;

R⁵ is selected from hydroxy, amino, C₁₋₆alkyl, C₁₋₆alkoxy, N,N—(C₁₋₆alkyl)₂amino, C₁₋₆alkoxycarbonylamino, carbocyclyl-R¹³— or heterocyclyl-R¹⁴—;

R¹³ and R¹⁴ are independently selected from a direct bond, —O—, or —N(R¹⁷)—; wherein R¹⁷ is hydrogen;

R⁶ is selected from C₁₋₆alkyl, C₁₋₆alkanoyl and C₁₋₆alkoxycarbonyl;

R³ is hydrogen;

R⁴ is selected from halo and C₁₋₆alkyl;

n is 1 or 2; wherein the values of R⁴ are the same or different;

or a pharmaceutically acceptable salt thereof.

Therefore in a further aspect of the invention there is provided a compound of formula (I) (as depicted above) wherein:

one of R¹ and R² is selected from C₁₋₆alkyl, C₂₋₆alkynyl, carbocyclyl or carbon-linked heterocyclyl; wherein this R¹ or R² may be optionally substituted on carbon by one or more R⁵; and wherein if said heterocyclyl contains an —NH— moiety that nitrogen may be optionally substituted by a group selected from R⁶; and

the other R¹ or R² is selected from C₁₋₆alkoxy;

R³ is hydrogen;

R⁴ is selected from halo and C₁₋₆alkyl;

n is 1 or 2; wherein the values of R⁴ are the same or different;

R⁵ is selected from hydroxy, amino, C₁₋₆alkyl, C₁₋₆alkoxy, N,N—(C₁₋₆alkyl)₂amino, C₁₋₆alkoxycarbonylamino, carbocyclyl-R¹³— or heterocyclyl-R¹⁴—;

R¹³ and R¹⁴ are independently selected from a direct bond, —O—, —N(R¹⁷)—; wherein R¹⁷ is hydrogen;

R⁶ is selected from C₁₋₆alkyl, C₁₋₆alkanoyl, C₁₋₆alkoxycarbonyl; wherein R⁶ may be optionally substituted on carbon by one or more R²²; and

R²² is selected from hydroxy or methoxy;

or a pharmaceutically acceptable salt thereof.

Therefore in a further aspect of the invention there is provided a compound of formula (I) (as depicted above) wherein:

One of R¹ and R² is selected from 3-hydroxypropyl, 3-piperidin-1-ylpropyl, 3-(cyclopropylamino)propyl, 3-dimethylaminopropyl, 3-aminopropyl, 3-(t-butoxycarbonylamino)propyl, 3-(3,4,5,6-tetrahydropyran-2-yloxy)propyl, cyclopropyl, 3-hydroxyprop-1-ynyl, pyridin-3-yl, 3,5-dimethylisoxazol-4-yl, pyrrol-2-yl, pyrimidin-5-yl, pyridin-4-yl, pyrazol-4-yl, 1-(t-butoxycarbonyl)-1,2,3,6-tetrahydropyridin-4-yl and 1-isobutylpyrazol-4-yl; and

the other R¹ or R² is selected from methoxy or ethoxy;

R³ is hydrogen;

R⁴ is selected from fluoro, chloro and ethyl; and

n is 1 or 2; wherein the values of R⁴ are the same or different;

or a pharmaceutically acceptable salt thereof.

Therefore in a further aspect of the invention there is provided a compound of formula (I) (as depicted above) wherein:

one of R¹ and R² is selected from 1,2,3,6-tetrahydropyridin-4-yl,
1-acetyl-3,6-dihydro-2H-pyridin-4-yl, 1H-pyrazol-4-yl, 1H-pyrrol-2-yl, 1-isobutylpyrazol-4-yl, 1-isopropyl-4-piperidyl, 1-methyl-4-piperidyl, 1-tert-butoxycarbonyl-3,6-dihydro-2H-pyridin-4-yl, 3-(1-piperidyl)propyl, 3-(cyclopropylamino)propyl, 3,5-dimethylisoxazol-4-yl, 3-aminopropyl, 3-dimethylaminopropyl, 3-hydroxyprop-1-ynyl, 3-hydroxypropyl, 3-pyridyl, 4-piperidyl, 4-pyridyl, 6-methoxy-3-pyridyl, 6-oxo-1H-pyridin-3-yl, cyclopropyl and pyrimidin-5-yl;

the other R¹ or R² is selected from methoxy and ethoxy;

R³ is hydrogen;

R⁴ is selected from fluoro, chloro, methyl and ethyl;

n is 1 or 2; wherein the values of R⁴ are the same or different;

or a pharmaceutically acceptable salt thereof.

Therefore in a further aspect of the invention there is provided a compound of formula (I) (as depicted above) wherein:

one of R¹ and R² is selected from 1-(2-hydroxyethyl)-4-piperidyl, 1-(3-methoxypropanoyl)-4-piperidyl, 1,2,3,6-tetrahydropyridin-4-yl, 1-[(2R)-2-hydroxypropanoyl]-4-piperidyl, 1-acetyl-3,6-dihydro-2H-pyridin-4-yl, 1-acetyl-4-piperidyl, 1H-pyrazol-4-yl, 1H-pyrrol-2-yl, 1-isobutylpyrazol-4-yl, 1-isopropyl-4-piperidyl, 1-methyl-4-piperidyl, 1-tert-butoxycarbonyl-3,6-dihydro-2H-pyridin-4-yl, 3-(1-piperidyl)propyl, 3-(cyclopropylamino)propyl, 3,5-dimethylisoxazol-4-yl, 3-aminopropyl, 3-dimethylaminopropyl, 3-hydroxyprop-1-ynyl, 3-hydroxypropyl, 3-pyridyl, 4-piperidyl, 4-pyridyl, 6-methoxy-3-pyridyl, 6-oxo-1H-pyridin-3-yl, cyclopropyl, pyrimidin-5-yl, 3-(t-butoxycarbonylamino)propyl or 3-(tetrahydro-2H-pyran-2-yloxy)propyl;

the other R¹ or R² is selected from methoxy or ethoxy.

R³ is hydrogen;

R⁴ is selected from fluoro, chloro, methyl and ethyl;

n is 1 or 2; wherein the values of R⁴ are the same or different;

or a pharmaceutically acceptable salt thereof.

In another aspect of the invention, preferred compounds of the invention are any one of the Examples or a pharmaceutically acceptable salt thereof.

In another aspect of the invention, preferred compounds of the invention are any one of Examples 42, 43, 46, 47, 49, 50, 51, 52, 53, 54 or a pharmaceutically acceptable salt thereof.

Another aspect of the present invention provides a process for preparing a compound of formula (I) or a pharmaceutically acceptable salt thereof which process (wherein variable groups are, unless otherwise specified, as defined in formula (I)) comprises of:

Process a) reacting a compound of formula (II):

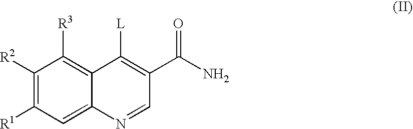

wherein L is a displaceable atom or group; with a compound of formula (III):

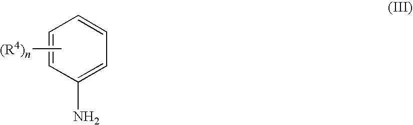

or
Process b) reacting a compound of formula (IV):

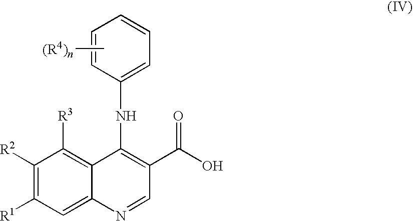

or an activated derivative thereof, with ammonia; or
Process c) reacting a compound of formula (V):

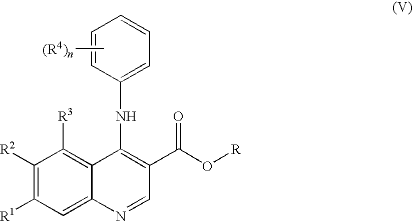

wherein R is C₁₋₆alkyl, in particular methyl and ethyl; with formamide and a base;
or
Process d) hydrolysis of a compound of formula (VI):

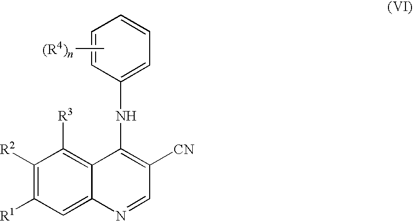

or
Process e) for compounds of formula (I) when one of R¹ and R² is selected from C₁₋₆alkyl, C₂₋₆alkenyl, C₂₋₆alkynyl, carbocyclyl or carbon-linked heterocyclyl, optionally substituted as stated herein above; by reaction of a compound of formula (VIIa) or (VIIb):

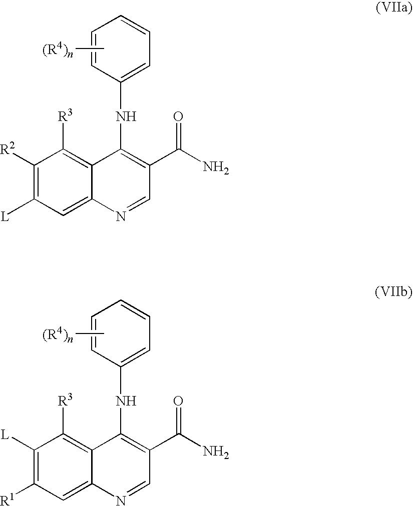

wherein L is a displaceable group; with a compound of formula (VIIIa) or (VIIIb):

R¹—B(R^{a})₂ (VIIIa)

R²—B(R^{a})₂ (VIIIb)

wherein —B(R^{a})₂ is a boronic acid derivative or trialkylborane; and thereafter if necessary:

i) converting a compound of the formula (I) into another compound of the formula (I);
ii) removing any protecting groups;
iii) forming a pharmaceutically acceptable salt.

L is a displaceable group, suitable values for L include chloro, bromo, tosyl and trifluoromethylsulphonyloxy.

—B(R^{a})₂ is a boronic acid derivative, suitable examples of boronic acid derivatives include dihydroxyboryl, 4,4,5,5-tetramethyl-1,3,2-dioxaborolanyl; a suitable example of a triakylborane is 9-borabicyclo[3.3.1]nonyl.

Specific reaction conditions for the above reactions are as follows.

Process a) Compounds of formula (II) can be reacted with compounds of formula (III) in a solvent such as ethanol or dimethylformamide, usually under thermal conditions often in the range of 70° C. to 100° C., and in some cases catalysed by the addition of acetic acid.

Alternatively, compounds of formula (II) can be reacted with compounds of formula (III) using coupling chemistry utilizing an appropriate catalyst and ligand such as Pd₂(dba)₃ and BINAP respectively and a suitable base such as sodium tert-butoxide or cesium carbonate. The reaction usually requires thermal conditions often in the range of 80° C. to 100° C.

Compounds of formula (II) may be prepared by a modification of Scheme 1 (see below).

Compounds of formula (III) are commercially available compounds or they are literature compounds or they are readily prepared by processes known to the person skilled in the art.

Process b) Acids of formula (IV) and ammonia may be coupled together in the presence of a suitable coupling reagent. Standard peptide coupling reagents known in the art can be employed as suitable coupling reagents, for example carbonyldiimidazole and dicyclohexyl-carbodiimide, optionally in the presence of a catalyst such as dimethylaminopyridine or 4-pyrrolidinopyridine, optionally in the presence of a base for example triethylamine, pyridine, or 2,6-di-alkyl-pyridines such as 2,6-lutidine or 2,6-di-tert-butylpyridine. Suitable solvents include dimethylacetamide, dichloromethane, benzene, tetrahydrofuran and dimethylformamide. The coupling reaction may conveniently be performed at a temperature in the range of −40 to 40° C.

Suitable activated acid derivatives include acid halides, for example acid chlorides, and active esters, for example pentafluorophenyl esters. The reaction of these types of compounds with amines is well known in the art, for example they may be reacted in the presence of a base, such as those described above, and in a suitable solvent, such as those described above. The reaction may conveniently be performed at a temperature in the range of −40 to 40° C.

Compounds of formula (IV) may be prepared by a modification of Scheme 1 (see below).

Process c) Esters of formula (V) may be reacted together with formamide and a base. Preferably this reaction occurs sequentially, addition of the formamide first, followed by the base. Suitable bases are alkoxide bases, for example methoxide and ethoxide bases, eg sodium methoxide. The reaction is typically performed at a temperature of 100° C. in a suitable solvent such as DMF.

Compounds of formula (V) may be prepared according to Scheme 1.

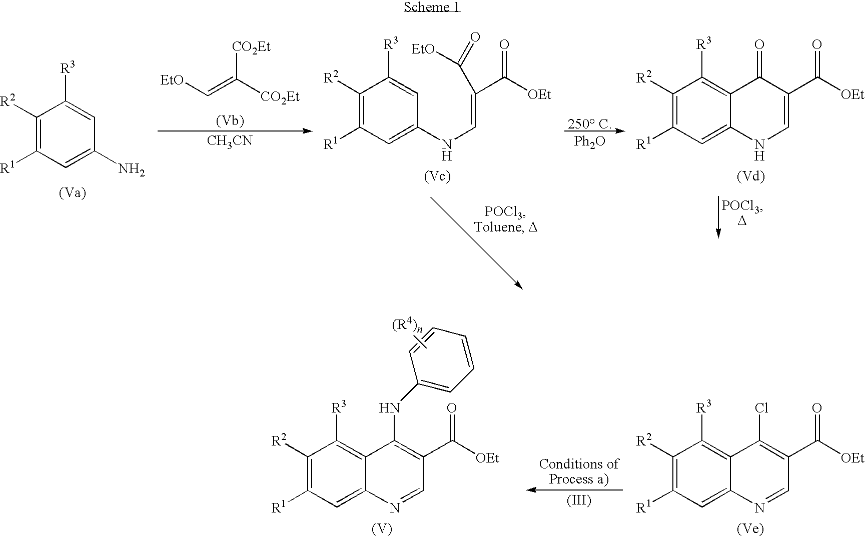

Compounds of formula (Va) and (Vb) are commercially available compounds or they are literature compounds or they are readily prepared by processes known to the person skilled in the art.

Process d) Compounds of formula (VI) can be hydrolysed under standard acidic or basic conditions.

Compounds of formula (VI) may be prepared by a modification of Scheme 1.

Process e) Compounds of formula (VIIa) and (VIIb) can be reacted with boronic acid derivatives of formula (VIIIa) and (VIIIb) using a palladium catalyst and a base. A suitable catalyst is Pd(PPh₃)₄ and a suitable base is potassium carbonate. The reaction is typically performed at a temperature of 100° C., or under microwave conditions, in a suitable solvent system such as dioxane/water.

Compounds of formula (VIIa) and (VIIb) can be reacted with trialkylboranes of formula (VIIIa) and (VIIIb) under standard Suzuki conditions, for example using a Pd catalyst in the presence of a base in a suitable solvent, for example, DMF typically at 50° C.

Compounds of formula (VIIa) and (VIIb) may be prepared by a modification of Scheme 1.

Compounds of formula (VIIIa) and (VIIb) are commercially available compounds or they are literature compounds or they are readily prepared by processes known to the person skilled in the art.

It will be appreciated that certain of the various ring substituents in the compounds of the present invention may be introduced by standard aromatic substitution reactions or generated by conventional functional group modifications either prior to or immediately following the processes mentioned above, and as such are included in the process aspect of the invention. Such reactions and modifications include, for example, introduction of a substituent by means of an aromatic substitution reaction, reduction of substituents, alkylation of substituents and oxidation of substituents. The reagents and reaction conditions for such procedures are well known in the chemical art. Particular examples of aromatic substitution reactions include the introduction of a nitro group using concentrated nitric acid, the introduction of an acyl group using, for example, an acyl halide and Lewis acid (such as aluminium trichloride) under Friedel Crafts conditions; the introduction of an alkyl group using an alkyl halide and Lewis acid (such as aluminium trichloride) under Friedel Crafts conditions; and the introduction of a halo group. Particular examples of modifications include the reduction of a nitro group to an amino group by for example, catalytic hydrogenation with a nickel catalyst or treatment with iron in the presence of hydrochloric acid with heating; oxidation of alkylthio to alkylsulphinyl or alkylsulphonyl.

It will also be appreciated that in some of the reactions mentioned herein it may be necessary/desirable to protect any sensitive groups in the compounds. The instances where protection is necessary or desirable and suitable methods for protection are known to those skilled in the art. Conventional protecting groups may be used in accordance with standard practice (for illustration see T. W. Green, Protective Groups in Organic Synthesis, John Wiley and Sons, 1991). Thus, if reactants include groups such as amino, carboxy or hydroxy it may be desirable to protect the group in some of the reactions mentioned herein.

A suitable protecting group for an amino or alkylamino group is, for example, an acyl group, for example an alkanoyl group such as acetyl, an alkoxycarbonyl group, for example a methoxycarbonyl, ethoxycarbonyl or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed for example, by hydrolysis with a suitable base such as an alkali metal hydroxide, for example lithium or sodium hydroxide. Alternatively an acyl group such as a t-butoxycarbonyl group may be removed, for example, by treatment with a suitable acid as hydrochloric, sulphuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium or sodium hydroxide. Alternatively an arylmethyl group such as a benzyl group may be removed, for example, by hydrogenation over a catalyst such as palladium-on-carbon.

A suitable protecting group for a carboxy group is, for example, an esterifying group, for example a methyl or an ethyl group which may be removed, for example, by hydrolysis with a base such as sodium hydroxide, or for example a t-butyl group which may be removed, for example, by treatment with an acid, for example an organic acid such as trifluoroacetic acid, or for example a benzyl group which may be removed, for example, by hydrogenation over a catalyst such as palladium-on-carbon.

The protecting groups may be removed at any convenient stage in the synthesis using conventional techniques well known in the chemical art.

Certain intermediates described herein are novel and these are provided as a further feature of the invention.

As stated hereinbefore the compounds defined in the present invention possess anti-cancer activity which is believed to arise from the CSF-1R kinase inhibitory activity of the compounds. These properties may be assessed, for example, using the procedure set out below.

### Biological Activity

### Assay 1: CSF-1R in Vitro AlphaScreen Assay

Activity of purified CSF-1R was determined in vitro using an Amplified Luminescent Proximity Homogeneous Assay (ALPHA)(Perkin Elmer), which measures phosphorylation of the CSF-1R substrate, biotinylated poly-glutamine-tyrosine peptide (PEY-HTRF CisBio 61GT0BLD), as described below. The His-tagged kinase domain of CSF-1R (i.e., amino acids 568-912, GeneBank ID NM_{—}005211; (see page 25 lines 13-19 of WO 2006/067445 for the sequence listing)) was purified from baculovirus infected SF+Express insect cells (1.4×106 cells/ml), French pressed and chromatographed through subsequent Qiagen Ni-NTA, Superflow Mono Q HR 10/10, and Superdex 200 SEC columns. Typical yield was 245 μg/l of cell pellet at >95% purity.

The phosphorylation of the CSF-1R substrate in the presence and absence of the compound of interest was determined. Briefly, 0.57 nM of purified CSF-1R, 5 nM pEY substrate, and compound were preincubated in 1× buffer for 30 minutes at 25° C. Reactions were initiated with addition of 90 μM adenosine triphosphate (ATP) in 1× buffer and incubated at 25° C. for 60 minutes and reactions stopped by addition of 5 μl of detection mix consisting of 136 mM NaCl, 102 mM ethylenediamine tetraacetic acid, 1.65 mg/ml BSA, 40 ug/ml Streptavidin donor beads (Perkin Elmer 6760002), 40 ug/ml pTyr100 acceptor beads (Perkin Elmer 6760620). Plates were incubated at 25° C. for 18 hours in the dark. Phosphorylated substrate was detected by an EnVision plate reader (Perkin Elmer) 680 nm excitation, 520-620 nm emission. Data was graphed and IC₅₀s calculated using Excel Fit (Microsoft).

Assay 2: CSF1R in-Vitro AlphaScreen Assay

Activity of purified CSF-1R was determined in-vitro using an Amplified Luminescent Proximity Homogeneous Assay (ALPHA) (Perkin Elmer, Mass.), which measures phosphorylation of CSF-1R substrate, biotinylated poly-glutamine-tyrosine peptide (pEY-HTRF CisBio 61GT0BLD), as described below. The His-tagged kinase domain of CSF-1R (i.e., amino acids 568-912, GeneBank ID NM_{—}005211) was purified from baculovirus infected SF+Express insect cells (1.4×106 cells/ml), French pressed and chromatographed through subsequent QIAgen Ni-NTA, Superflow Mono Q HR 10/10, and Superdex 200 SEC columns. Typical yield was 322 ug/l of cell pellet at >95% purity. The phosphorylation of the CSF-1R substrate in the presence and absence of the compound of interest was determined. Briefly, 5 ul of Enzyme/Substrate/adenosine triphosphate (ATP) mix consisting of 0.46 nM of purified CSF-1R, 12 nM pEY substrate, and 12 mM ATP in 1.2× buffer was preincubated with 2 ul of compound for 20 minutes at 25° C. Reactions were initiated with 5 ul of Metal mix consisting of 24 mM MgCl₂ in 1.2× buffer and incubated at 25° C. for 90 minutes and reactions were stopped by addition of 5 ul of Detection mix consisting of 20 mM HEPES, 102 mM ethylenediamine tetraacetic acid, 1.65 mg/ml BSA, 136 mM NaCl, 40 ug/ml Streptavidin donor beads (Perkin Elmer, Mass., Catalog #6760002), and 40 ug/ml phosphotyrosine-specific antibody coated acceptor beads (Perkin Elmer, Mass., Catalog #6760620). Plates were incubated at 25° C. for 18 hours in the dark. Phosphorylated substrate was detected by an EnVision plate reader (Perkin Elmer) 680 nm excitation, 520-620 nm emission. Data was graphed and IC₅₀s calculated using Excel Fit (Microsoft). When tested in one or other of the above in vitro assays, the compounds of the present invention generally exhibited activity less than 30 μM. For example the following results were obtained in an assay substantially similar to one or other of the assays described hereinabove:

| Ex | Assay 1 IC₅₀ (μM) | Assay 2 IC₅₀ (μM) |
|---|---|---|
| 1 | 0.002 | |
| 3 | 0.005 | |
| 8 | 0.020 | |
| 9 | 0.003 | |
| 10 | 0.023 | |
| 11 | 0.004 | |
| 12 | 0.004 | |
| 13 | 0.001 | |
| 14 | 0.008 | |
| 15 | 0.002 | |
| 16 | 0.005 | |
| 17 | 0.009 | |
| 18 | <0.003 | |
| 19 | 0.008 | |
| 20 | 0.038 | |
| 21 | 0.018 | |
| 22 | 0.033 | |
| 23 | 0.022 | |
| 24 | 0.026 | |
| 25 | 0.007 | |
| 26 | 0.140 | |
| 27 | 0.065 | |
| 28 | 0.014 | |
| 29 | 0.057 | |
| 30 | 0.076 | |
| 31 | 0.043 | |
| 32 | 0.035 | |
| 33 | 0.096 | |
| 34 | 0.025 | |
| 35 | 0.061 | |
| 36 | 0.028 | |
| 37 | 0.440 | |
| 38 | 0.660 | |
| 39 | <0.003 | |
| 40 | 0.007 | |
| 41 | 0.014 | |
| 42 | 0.005 | |
| 43 | <0.0039 | |
| 44 | <0.003 | |
| 45 | <0.003 | |
| 46 | 0.018 | 0.022 |
| 47 | 0.004 | |
| 48 | 0.003 | |
| 49 | 0.007 | |
| 50 | 0.006 | |
| 51 | 0.010 | |
| 52 | | 0.014 |
| 53 | | 0.032 |
| 54 | | 0.008 |
| 55 | 0.015 | |
| 56 | | 0.030 |
| 57 | | 0.071 |
| 58 | | 0.052 |
| 59 | 0.007 | |
| 60 | | 0.004 |

According to a further aspect of the invention there is provided a pharmaceutical composition which comprises a compound of the formula (I), or a pharmaceutically acceptable salt thereof, as defined hereinbefore, in association with a pharmaceutically-acceptable diluent or carrier.

The composition may be in a form suitable for oral administration, for example as a tablet or capsule, for parenteral injection (including intravenous, subcutaneous, intramuscular, intravascular or infusion) as a sterile solution, suspension or emulsion, for topical administration as an ointment or cream or for rectal administration as a suppository.

In general the above compositions may be prepared in a conventional manner using conventional excipients.

The compound of formula (I) will normally be administered to a warm-blooded animal at a unit dose within the range 1-1000 mg/kg, and this normally provides a therapeutically-effective dose. Preferably a daily dose in the range of 10-100 mg/kg is employed. However the daily dose will necessarily be varied depending upon the host treated, the particular route of administration, and the severity of the illness being treated. Accordingly the optimum dosage may be determined by the practitioner who is treating any particular patient.

According to a further aspect of the present invention there is provided a compound of the formula (I), or a pharmaceutically acceptable salt thereof, as defined hereinbefore for use in a method of treatment of the human or animal body by therapy.

We have found that the compounds defined in the present invention, or a pharmaceutically acceptable salt thereof, are effective anti-cancer agents which property is believed to arise from their CSF-1R kinase inhibitory properties. Accordingly the compounds of the present invention are expected to be useful in the treatment of diseases or medical conditions mediated alone or in part by CSF-1R kinase, i.e. the compounds may be used to produce a CSF-1R kinase inhibitory effect in a warm-blooded animal in need of such treatment.

Thus the compounds of the present invention provide a method for treating cancer characterised by inhibition of CSF-1R kinase, i.e. the compounds may be used to produce an anti-cancer effect mediated alone or in part by the inhibition of CSF-1R kinase.

Such a compound of the invention is expected to possess a wide range of anti-cancer properties as aberrant expression of CSF1R and/or CSF1 has been observed in multiple human cancers and derived cell lines, including but not limited to, breast, ovarian, endometrial, prostate, lung, kidney and pancreatic tumors as well as haematological malignancies including, but not limited to, myelodysplastic syndrome, acute myelogenous leukemia, chronic myelogenous leukemia, non Hodgkin's lymphoma, Hodgkin's disease, multiple myeloma and chronic lymphocytic leukemia. Activating mutations have also been reported in haematopoietic and lymphoid tissue and lung cancer. Further, tumor associated macrophages have been associated with poor prognosis in multiple tumor types including, but not limited to, breast, endometrial, kidney, lung, bladder and cervical cancers, glioma, squamous cell carcinoma of the esophagus, malignant uveal melanoma and follicular lymphoma. It is expected that a compound of the invention will possess anticancer activity against these cancers through direct effect on the tumor and/or indirectly through effect on tumor associated macrophages. Particularly the cancer is breast cancer. In another aspect of the invention, particularly the cancer is ovarian cancer.

In a further aspect of the invention, compounds of formula (I) may be also be of value in the treatment of certain additional indications. These indications include, but are not limited to tumor-associated osteolysis, osteoporosis including ovariectomy-induced bone loss, orthopedic implant failure, autoimmune disorders including systemic lupus erythematosus, arthritis including rheumatoid arthritis, osteoarthritis, renal inflammation and glomerulonephritis; inflammatory bowel disease; transplant rejection including renal and bone marrow allografts and skin xenograft, atherosclerosis, obesity, Alzheimer's Disease and Langerhans cell histiocytosis. A further aspect of the present invention therefore includes the treatment of one of more of these diseases, particularly arthritis including rheumatoid arthritis and osteoarthritis. These indications also include, but are not limited to chronic obstructive pulmonary disease, diabetes and chronic skin disorders including psoriasis. Particularly this indication is osteoarthritis. In another aspect of the invention, particularly this indication is rheumatoid arthritis.

Thus according to this aspect of the invention there is provided a compound of the formula (I), or a pharmaceutically acceptable salt thereof, as defined hereinbefore for use as a medicament.

According to a further aspect of the invention there is provided the use of a compound of the formula (I), or a pharmaceutically acceptable salt thereof, as defined hereinbefore in the manufacture of a medicament for use in the production of a CSF-1R kinase inhibitory effect in a warm-blooded animal such as man.

According to this aspect of the invention there is provided the use of a compound of the formula (I), or a pharmaceutically acceptable salt thereof, as defined hereinbefore in the manufacture of a medicament for use in the production of an anti-cancer effect in a warm-blooded animal such as man.

According to a further feature of the invention, there is provided the use of a compound of the formula (I), or a pharmaceutically acceptable salt thereof, as defined herein before in the manufacture of a medicament for use in the treatment of breast, ovarian, bladder, cervical, endometrial, prostate, lung, kidney and pancreatic tumors; haematological malignancies including myelodysplastic syndrome, acute myelogenous leukemia, chronic myelogenous leukemia, non Hodgkin's lymphoma, Hodgkin's disease, multiple myeloma and chronic lymphocytic leukemia; and glioma, squamous cell carcinoma of the esophagus, malignant uveal melanoma and follicular lymphoma.

According to a further feature of the invention, there is provided the use of a compound of the formula (I), or a pharmaceutically acceptable salt thereof, as defined herein before in the manufacture of a medicament for use in the treatment of tumor-associated osteolysis, osteoporosis including ovariectomy-induced bone loss, orthopedic implant failure, autoimmune disorders including systemic lupus erythematosus, arthritis including rheumatoid arthritis, osteoarthritis, renal inflammation and glomerulonephritis; inflammatory bowel disease; transplant rejection including renal and bone marrow allografts and skin xenograft, atherosclerosis, obesity, Alzheimer's Disease, chronic obstructive pulmonary disease, diabetes and chronic skin disorders including psoriasis and Langerhans cell histiocytosis

According to a further feature of this aspect of the invention there is provided a method for producing a CSF-1R kinase inhibitory effect in a warm-blooded animal, such as man, in need of such treatment which comprises administering to said animal an effective amount of a compound of formula (I), or a pharmaceutically acceptable salt thereof, as defined above.

According to a further feature of this aspect of the invention there is provided a method for producing an anti-cancer effect in a warm-blooded animal, such as man, in need of such treatment which comprises administering to said animal an effective amount of a compound of formula (I), or a pharmaceutically acceptable salt thereof, as defined above.

According to an additional feature of this aspect of the invention there is provided a method of treating breast, ovarian, bladder, cervical, endometrial, prostate, lung, kidney and pancreatic tumors; haematological malignancies including myelodysplastic syndrome, acute myelogenous leukemia, chronic myelogenous leukemia, non Hodgkin's lymphoma, Hodgkin's disease, multiple myeloma and chronic lymphocytic leukemia; and glioma, squamous cell carcinoma of the esophagus, malignant uveal melanoma and follicular lymphoma in a warm-blooded animal, such as man, in need of such treatment which comprises administering to said animal an effective amount of a compound of formula (I) or a pharmaceutically acceptable salt thereof as defined herein before.

According to an additional feature of this aspect of the invention there is provided a method of treating tumor-associated osteolysis, osteoporosis including ovariectomy-induced bone loss, orthopedic implant failure, autoimmune disorders including systemic lupus erythematosus, arthritis including rheumatoid arthritis, osteoarthritis, renal inflammation and glomerulonephritis; inflammatory bowel disease; transplant rejection including renal and bone marrow allografts and skin xenograft, atherosclerosis, obesity, Alzheimer's Disease, chronic obstructive pulmonary disease, diabetes and chronic skin disorders including psoriasis and Langerhans cell histiocytosis in a warm-blooded animal, such as man, in need of such treatment which comprises administering to said animal an effective amount of a compound of formula (I) or a pharmaceutically acceptable salt thereof as defined herein before.

In a further aspect of the invention there is provided a pharmaceutical composition which comprises a compound of the formula (I), or a pharmaceutically acceptable salt thereof, as defined herein before in association with a pharmaceutically-acceptable diluent or carrier for use in the production of a CSF-1R kinase inhibitory effect in a warm-blooded animal such as man.

In a further aspect of the invention there is provided a pharmaceutical composition which comprises a compound of the formula (I), or a pharmaceutically acceptable salt thereof, as defined herein before in association with a pharmaceutically-acceptable diluent or carrier for use in the production of an anti-cancer effect in a warm-blooded animal such as man.

In a further aspect of the invention there is provided a pharmaceutical composition which comprises a compound of the formula (I), or a pharmaceutically acceptable salt thereof, as defined herein before in association with a pharmaceutically-acceptable diluent or carrier for use in the treatment of breast, ovarian, bladder, cervical, endometrial, prostate, lung, kidney and pancreatic tumors; haematological malignancies including myelodysplastic syndrome, acute myelogenous leukemia, chronic myelogenous leukemia, non Hodgkin's lymphoma, Hodgkin's disease, multiple myeloma and chronic lymphocytic leukemia; and glioma, squamous cell carcinoma of the esophagus, malignant uveal melanoma and follicular lymphoma in a warm-blooded animal such as man.

In a further aspect of the invention there is provided a pharmaceutical composition which comprises a compound of the formula (I), or a pharmaceutically acceptable salt thereof, as defined herein before in association with a pharmaceutically-acceptable diluent or carrier for use in the treatment of tumor-associated osteolysis, osteoporosis including ovariectomy-induced bone loss, orthopedic implant failure, autoimmune disorders including systemic lupus erythematosus, arthritis including rheumatoid arthritis, osteoarthritis, renal inflammation and glomerulonephritis; inflammatory bowel disease; transplant rejection including renal and bone marrow allografts and skin xenograft, atherosclerosis, obesity, Alzheimer's Disease, chronic obstructive pulmonary disease, diabetes and chronic skin disorders including psoriasis and Langerhans cell histiocytosis in a warm-blooded animal such as man.

According to a further aspect of the invention there is provided the use of a compound of the formula (I), or a pharmaceutically acceptable salt thereof, as defined hereinbefore in the production of a CSF-1R kinase inhibitory effect in a warm-blooded animal such as man.

According to this aspect of the invention there is provided the use of a compound of the formula (I), or a pharmaceutically acceptable salt thereof, as defined hereinbefore in the production of an anti-cancer effect in a warm-blooded animal such as man.

According to a further feature of the invention, there is provided the use of a compound of the formula (I), or a pharmaceutically acceptable salt thereof, as defined herein before in the treatment of breast, ovarian, bladder, cervical, endometrial, prostate, lung, kidney and pancreatic tumors; haematological malignancies including myelodysplastic syndrome, acute myelogenous leukemia, chronic myelogenous leukemia, non Hodgkin's lymphoma, Hodgkin's disease, multiple myeloma and chronic lymphocytic leukemia; and glioma, squamous cell carcinoma of the esophagus, malignant uveal melanoma and follicular lymphoma.

According to a further feature of the invention, there is provided the use of a compound of the formula (I), or a pharmaceutically acceptable salt thereof, as defined herein before in the treatment of tumor-associated osteolysis, osteoporosis including ovariectomy-induced bone loss, orthopedic implant failure, autoimmune disorders including systemic lupus erythematosus, arthritis including rheumatoid arthritis, osteoarthritis, renal inflammation and glomerulonephritis; inflammatory bowel disease; transplant rejection including renal and bone marrow allografts and skin xenograft, atherosclerosis, obesity, Alzheimer's Disease, chronic obstructive pulmonary disease, diabetes and chronic skin disorders including psoriasis and Langerhans cell histiocytosis.

A compound of the formula (I), or a pharmaceutically acceptable salt thereof, as defined herein before, for the production of a CSF-1R kinase inhibitory effect in a warm-blooded animal such as man.

A compound of the formula (I), or a pharmaceutically acceptable salt thereof, as defined herein before, for the production of an anti-cancer effect in a warm-blooded animal such as man.

A compound of the formula (I), or a pharmaceutically acceptable salt thereof, as defined herein before, for the treatment of melanoma, papillary thyroid tumours, cholangiocarcinomas, colon cancer, ovarian cancer, lung cancer, leukemias, lymphoid malignancies, carcinomas and sarcomas in the liver, kidney, bladder, prostate, breast and pancreas, and primary and recurrent solid tumours of the skin, colon, thyroid, lungs and ovaries.

A compound of the formula (I), or a pharmaceutically acceptable salt thereof, as defined herein before, for the treatment of breast, ovarian, bladder, cervical, endometrial, prostate, lung, kidney and pancreatic tumors; haematological malignancies including myelodysplastic syndrome, acute myelogenous leukemia, chronic myelogenous leukemia, non Hodgkin's lymphoma, Hodgkin's disease, multiple myeloma and chronic lymphocytic leukemia; and glioma, squamous cell carcinoma of the esophagus, malignant uveal melanoma and follicular lymphoma.

A compound of the formula (I), or a pharmaceutically acceptable salt thereof, as defined herein before, for the treatment of tumor-associated osteolysis, osteoporosis including ovariectomy-induced bone loss, orthopedic implant failure, autoimmune disorders including systemic lupus erythematosus, arthritis including rheumatoid arthritis, osteoarthritis, renal inflammation and glomerulonephritis; inflammatory bowel disease; transplant rejection including renal and bone marrow allografts and skin xenograft, atherosclerosis, obesity, Alzheimer's Disease, chronic obstructive pulmonary disease, diabetes and chronic skin disorders including psoriasis and Langerhans cell histiocytosis.

The CSF-1R kinase inhibitory treatment defined hereinbefore may be applied as a sole therapy or may involve, in addition to the compound of the invention, conventional surgery or radiotherapy or chemotherapy. Such chemotherapy may include one or more of the following categories of anti-tumour agents:—

(i) antiproliferative/antineoplastic drugs and combinations thereof, as used in medical oncology, such as alkylating agents (for example cis-platin, carboplatin, cyclophosphamide, nitrogen mustard, melphalan, chlorambucil, busulphan and nitrosoureas); antimetabolites (for example antifolates such as fluoropyrimidines like 5-fluorouracil and tegafur, raltitrexed, methotrexate, cytosine arabinoside and hydroxyurea; antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere); and topoisomerase inhibitors (for example epipodophyllotoxins like etoposide and teniposide, amsacrine, topotecan and camptothecin);
(ii) cytostatic agents such as antioestrogens (for example tamoxifen, toremifene, raloxifene, droloxifene and iodoxyfene), oestrogen receptor down regulators (for example fulvestrant), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5α-reductase such as finasteride;
(iii) agents which inhibit cancer cell invasion (for example metalloproteinase inhibitors like marimastat and inhibitors of urokinase plasminogen activator receptor function);
(iv) inhibitors of growth factor function, for example such inhibitors include growth factor antibodies, growth factor receptor antibodies (for example the anti-erbb2 antibody trastuzumab [Herceptin™] and the anti-erbb 1 antibody cetuximab [C225]), farnesyl transferase inhibitors, MEK inhibitors, tyrosine kinase inhibitors and serine/threonine kinase inhibitors, for example inhibitors of the epidermal growth factor family (for example EGFR family tyrosine kinase inhibitors such as N-(3-chloro-4-fluorophenyl)-7-methoxy-6-(3-morpholinopropoxy)quinazolin-4-amine (gefitinib, AZD1839), N-(3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)quinazolin-4-amine (CI 1033)), for example inhibitors of the platelet-derived growth factor family and for example inhibitors of the hepatocyte growth factor family;
(v) antiangiogenic agents such as those which inhibit the effects of vascular endothelial growth factor, (for example the anti-vascular endothelial cell growth factor antibody bevacizumab [Avastin™], compounds such as those disclosed in International Patent Applications WO 97/22596, WO 97/30035, WO 97/32856 and WO 98/13354) and compounds that work by other mechanisms (for example linomide, inhibitors of integrin αvβ3 function and angiostatin);
(vi) vascular damaging agents such as Combretastatin A4 and compounds disclosed in International Patent Applications WO 99/02166, WO00/40529, WO 00/41669, WO01/92224, WO02/04434 and WO02/08213;
(vii) antisense therapies, for example those which are directed to the targets listed above, such as ISIS 2503, an anti-ras antisense;
(viii) gene therapy approaches, including for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy;
(ix) immunotherapy approaches, including for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies;
(x) cell cycle inhibitors including for example CDK inhibitiors (eg flavopiridol) and other inhibitors of cell cycle checkpoints (eg checkpoint kinase); inhibitors of aurora kinase and other kinases involved in mitosis and cytokinesis regulation (eg mitotic kinesins); and histone deacetylase inhibitors; and
(xi) endothelin antagonists, including endothelin A antagonists, endothelin B antagonists and endothelin A and B antagonists; for example ZD4054 and ZD1611 (WO 96 40681), atrasentan and YM598.

Therefore, in a further aspect of the invention there is provided a compound of the formula (I), or a pharmaceutically acceptable salt thereof and a chemotherapeutic agent selected from:

(i) one or more antiproliferative/antineoplastic drugs; and/or
(ii) one or more cytostatic agents; and/or
(iii) one or more agents which inhibit cancer cell invasion; and/or
(iv) one or more inhibitors of growth factor function; and/or
(v) one or more antiangiogenic agents; and/or
(vi) one or more vascular damaging agents; and/or
(vii) one or more antisense therapies; and/or
(viii) one or more gene therapy approaches; and/or
(ix) one or more immunotherapy approaches; and/or
(x) one or more cell cycle inhibitors; and/or
(xi) one or more endothelin antagonists.

Such conjoint treatment may be achieved by way of the simultaneous, sequential or separate dosing of the individual components of the treatment. Such combination products employ the compounds of this invention within the dosage range described hereinbefore and the other pharmaceutically-active agent within its approved dosage range.

In addition to their use in therapeutic medicine, the compounds of formula (I) and their pharmaceutically acceptable salts are also useful as pharmacological tools in the development and standardisation of in vitro and in vivo test systems for the evaluation of the effects of inhibitors of CSF-1R kinase in laboratory animals such as cats, dogs, rabbits, monkeys, rats and mice, as part of the search for new therapeutic agents.

In the above other pharmaceutical composition, process, method, use and medicament manufacture features, the alternative and preferred embodiments of the compounds of the invention described herein also apply.

### EXAMPLES

The invention will now be illustrated by the following non limiting examples in which, unless stated otherwise:

(i) temperatures are given in degrees Celsius (° C.); operations were carried out at room or ambient temperature unless otherwise stated, that is, at a temperature in the range of 18-25° C.;
(ii) organic solutions were dried over anhydrous sodium sulphate or magnesium sulphate; evaporation of solvent was carried out using a rotary evaporator under reduced pressure (600-4000 Pascals; 4.5-30 mmHg) with a bath temperature of up to 60° C.;
(iii) in general, the course of reactions was followed by TLC and reaction times are given for illustration only;
(iv) final products had satisfactory proton nuclear magnetic resonance (NMR) spectra and/or mass spectral data;
(v) yields are given for illustration only and are not necessarily those which can be obtained by diligent process development; preparations were repeated if more material was required;
(vii) when given, NMR data is in the form of delta values for major diagnostic protons, given in parts per million (ppm) relative to tetramethylsilane (TMS) as an internal standard, determined at 400 MHz using perdeuterio dimethyl sulphoxide (DMSO-d₆) as solvent unless otherwise indicated;
(vii) chemical symbols have their usual meanings; SI units and symbols are used;
(viii) solvent ratios are given in volume:volume (v/v) terms; and
(ix) mass spectra were run with an electron energy of 70 electron volts in the chemical ionization (CI) mode using a direct exposure probe; where indicated ionization was effected by electron impact (EI), fast atom bombardment (FAB) or electrospray (ESP); values for m/z are given; generally, only ions which indicate the parent mass are reported; and unless otherwise stated, the mass ion quoted is (MH)⁺;
(x) where a synthesis is described as being analogous to that described in a previous example the amounts used are the millimolar ratio equivalents to those used in the previous example;
(xi) the following abbreviations have been used:

DMF N,N-dimethylformamide;

EtOAc ethyl acetate;

MeOH methanol;

THF tetrahydrofuran;

TBAF tetrabutylammonium fluoride;

TFA trifluoroacetic acid;

DMSO dimethylsulphoxide;

(xii) “ISCO” refers to normal phase flash column chromatography using 12 g and 40 g pre-packed silica gel cartridges used according to the manufacturers instruction obtained from ISCO, Inc, 4700 superior street Lincoln, Nebr., USA.;
(xiii) “Gilson” refers to a YMC-AQC18 reverse phase HPLC Column with dimension 20 mm/100 and 50 mm/250 in water/MeCN with 0.1% TFA as mobile phase; and
(xiv) “Berger SFC” refers to supercritical fluid chromatography using a Diol SFC column 21.2×250 mm with 40% methanol as modifier, flow rate 60 mls/min, 40° C., pressure 100 bar.

### Example 1

### 6-(3-Aminopropyl)-4-[(3,4-dichlorophenyl)amino]-7-methoxyquinoline-3-carboxamide

A solution of tert-butyl (3-{3-(aminocarbonyl)-4-[(3,4-dichlorophenyl)amino]-7-methoxyquinolin-6-yl}propyl)carbamate (Example 2; 500 mg, 0.96 mmol) in TFA:DCM (1:1, 10 mL) was stirred for 1 hour. The solvent was removed under reduced pressure, and the resulting oil was triturated with diethyl ether for 16 hours to give 204 mg solid. ¹H NMR: 10.94 (s, 1H), 8.89 (s, 1H), 8.17 (s, 1H), 7.98 (s, 1H), 7.77 (s, 2H), 7.71 (s, 1H), 7.58 (d, 1H), 7.42 (d, 1H), 4.01 (s, 3H), 2.81 (m, 2H), 2.71 (m, 2H), 1.80 (m, 2H); m/z: 420.

### Example 2

### tert-Butyl (3-{3-(aminocarbonyl)-4-[(3,4-dichlorophenyl)amino]-7-methoxyquinolin-6-yl}propyl)carbamate

To a solution of ethyl 6-{3-[(tert-butoxycarbonyl)amino]propyl}-4-[(3,4-dichlorophenyl)amino]-7-methoxyquinoline-3-carboxylate (Intermediate 1; 600 mg, 1.10 mmol) and formamide (350 μL, 8.8 mmol) in DMF (5 mL) at 100° C. under nitrogen was added dropwise over 10 minutes a solution of NaOMe (0.5 M in MeOH, 6.5 mL, 3.28 mmol). After 16 hours at 100° C., the reaction mixture was cooled, poured into brine (200 mL) and extracted with EtOAc (3×200 mL). The combined organic extracts were dried (Na₂SO₄), concentrated under reduced pressure, and the residue was subjected to normal phase chromatography on the ISCO eluting with EtOAc to give 500 mg of an oil. m/z: 548.

### Examples 3-7

The following compounds were prepared by a similar method to Example 2 using the appropriate starting materials.

| Ex | Name | NMR/m/z | SM |
|---|---|---|---|
| 3 | 4-[(3-Chloro-2- | CD₃OD 8.78 (s, 1 H), 7.43 (s, 1 H), 7.18 (s, | Intermediate |
| | fluorophenyl)amino]-7- | 1 H), 7.07 (m, 1 H), 6.92 (td, 1 H), 6.75 (m, | 69 |
| | ethoxy-6-(3- | 1 H), 4.15 (q, 2 H), 3.38 (t, 2 H), 2.55 (m, 2 | |
| | hydroxypropyl)quinoline- | H), 1.59 (m, 2 H), 1.41 (t, 3 H); m/z 418 | |
| | 3-carboxamide | | |
| 4 | 7-Ethoxy-4-[(4- | 10.80 (s, 1 H), 8.91 (s, 1 H), 8.22 (s, 1 H), | Intermediate |
| | ethylphenyl)amino]-6-[3- | 7.59 (s, 1 H), 7.35 (s, 1 H), 7.23 (s, 1 H), | 61 |
| | (tetrahydro-2H-pyran-2- | 7.12 (d, 2 H), 6.90 (d, 2 H), 4.45 (t, 1 H), | |
| | yloxy)propyl]quinoline-3- | 4.18 (q, 2 H), 3.65 (m, 1 H), 3.46 (m, 1 H), | |
| | carboxamide | 3.32 (m, 1 H), 3.17 (m, 1 H), 2.57 (q, 2 H), | |
| | | 2.50 (q, 2 H), 1.67 (m, 1 H), 1.38-1.55 (m, | |
| | | 10 H), 1.16 (t, 3 H) | |
| 5 | 4-[(3,4- | CD₃OD 8.85 (s, 1 H), 7.63 (s, 1 H), 7.38 (s, | Intermediate |
| | Dichlorophenyl)amino]-7- | 1 H), 7.28 (m, 1 H), 7.14 (d, 1 H), 6.91 (dd, | 62 |
| | ethoxy-6-[3-(tetrahydro- | 1 H), 4.53 (m, 1 H), 4.26 (q, 2 H), 3.80 (m, | |
| | 2H-pyran-2- | 1 H), 3.66 (dd, 1 H), 3.46 (m, 1 H), 3.27 | |
| | yloxy)propyl]quinoline-3- | (m, 1 H), 2.75 (t, 2 H), 1.74-1.85 (m, 3 H), | |
| | carboxamide | 1.67 (m, 1 H), 1.53 (m, 7 H) | |
| 6 | 4-[(2,3- | CD₃OD 8.92 (s, 1 H), 7.32 (s, 1 H), 7.24 (s, | Intermediate |
| | Dichlorophenyl)amino]-7- | 1 H), 7.19 (dd, 1 H), 7.04 (t, 1 H), 6.62 (d, | 63 |
| | ethoxy-6-[3-(tetrahydro- | 1 H), 4.47 (m, 1 H), 4.22 (q, 2 H), 3.78 (d, | |
| | 2H-pyran-2- | 1 H), 3.59 (m, 1 H), 3.45 (m, 1 H), 3.25 (m, | |
| | yloxy)propyl]quinoline-3- | 1 H), 2.64 (t, 2 H), 1.66-1.76 (m, 4 H), | |
| | carboxamide | 1.46-1.57 (m, 7 H) | |
| 7 | 4-[(3-Chloro-4- | 11.56 (s, 1 H), 8.99 (s, 1 H), 8.38 (s, 1 H), | Intermediate |
| | fluorophenyl)amino]-7- | 7.91 (s, 1 H), 7.81 (s, 1 H), 7.48-7.57 (m, | 64 |
| | ethoxy-6-[3-(tetrahydro- | 3 H), 7.32 (m, 1 H), 4.57 (m, 1 H), 4.29 (q, | |
| | 2H-pyran-2- | 2 H), 3.74 (m, 1 H), 3.62 (m, 1 H), 3.45 (m, | |
| | yloxy)propyl]quinoline-3- | 1 H), 3.36 (m, 1 H), 2.71 (m, 2 H), 1.76 (m, | |
| | carboxamide | 3 H), 1.65 (m, 1 H), 1.45-1.56 (m, 7 H) | |

### Example 8

### 4-[(2,4-Difluorophenyl)amino]-7-methoxy-6-(3-piperidin-1-ylpropyl)quinoline-3-carboxamide

To a solution of 4-[(2,4-difluorophenyl)amino]-6-(3-hydroxypropyl)-7-methoxyquinoline-3-carboxamide (Example 14; 21 mg, 0.54 mmol) and triethylamine (735 μL 5.4 mmol) in THF (15 mL) at 0° C. was added dropwise over 5 minutes a solution of mesyl chloride (42 μL, 0.52 mmol) in THF (1 mL). After warming to RT over 1 hour, the reaction mixture was used in subsequent reactions.

To approximately one third of the above reaction mixture was added piperidine (532 μL, 5.4 mmol). After 16 hours, the solvent was removed under reduced pressure, and the residue partitioned between saturated potassium carbonate solution (20 mL) and EtOAc (20 mL). The aqueous phase was extracted with EtOAc (3×30 mL), and the combined organic extracts were dried (Na₂SO₄), filtered, and concentrated under reduced pressure to give 17 mg of an oil. ¹H NMR (CD₃OD): 8.76 (s, 1H), 7.37 (s, 1H), 7.18 (s, 1H), 6.99 (m, 2H), 6.82 (m, 1H), 3.90 (s, 3H), 2.55 (m, 4H), 2.48 (t, 2H), 2.41 (t, 2H), 1.59 (m, 6H), 1.45 (m, 2H); m/z: 455.

### Examples 9-13

The following compounds were prepared by methods similar to those for Example 8 using the appropriate starting materials.

| Ex | Name | NMR | MS | SM |
|---|---|---|---|---|
| 9 | 4-[(2,4-Difluorophenyl)amino]-6- | CD₃OD 8.76 (s, 1 H), 7.71 (s, 1 H), | 415 | Example |
| | [3-(dimethylamino)propyl]-7- | 7.36 (m, 1 H), 7.24 (s, 1 H), 7.13 | | 14 |
| | methoxyquinoline-3-carboxamide | (m, 1 H), 7.02 (m, 1 H), 4.00 (s, 3 | | |
| | | H), 3.04 (t, 2 H), 2.79 (s, 6 H), 2.62 | | |
| | | (m, 2 H), 1.82 (m, 2 H) | | |
| 10 | 6-[3-(Cyclopropylamino)propyl]- | CD₃OD 8.75 (s, 1 H), 7.43 (s, 1 H), | 427 | Example |
| | 4-[(2,4-difluorophenyl)amino]-7- | 7.20 (s, 1 H), 6.98 (m, 2 H), 6.82 | | 14 |
| | methoxyquinoline-3-carboxamide | (m, 1 H), 3.90 (s, 3 H), 2.87 (t, 2 | | |
| | | H), 2.55 (m, 3 H), 1.78 (m, 2 H), | | |
| | | 0.74 (m, 4 H) | | |
| 11 | 6-[3-(Cyclopropylamino)propyl]- | CD₃OD 8.72 (s, 1 H), 8.01 (s, 1 H), | 459 | Example |
| | 4-[(3,4-dichlorophenyl)amino]-7- | 7.48 (m, 2 H), 7.28 (s, 1 H), 7.20 | | 15 |
| | methoxyquinoline-3-carboxamide | (m, 1 H), 4.01 (s, 3 H), 2.99 (m, 2 | | |
| | | H), 2.65 (m, 3 H), 1.94 (m, 2 H), | | |
| | | 0.83 (m, 4 H) | | |
| 12 | 4-[(3,4-Dichlorophenyl)amino]-7- | CD₃OD 8.86 (s, 1 H), 7.99 (s, 1 H), | 487 | Example |
| | methoxy-6-(3-piperidin-1- | 7.63 (d, 1 H), 7.57 (s, 1 H), 7.34 (s, | | 15 |
| | ylpropyl)quinoline-3-carboxamide | 1 H), 7.29 (d, 1 H), 4.11 (s, 3 H), | | |
| | | 3.51 (m, 2 H), 3.15 (m, 2 H), 2.94 | | |
| | | (t, 2 H), 2.77 (t, 2 H), 1.85 (m, 8 H) | | |
| 13 | 4-[(3,4-Dichlorophenyl)amino]-6- | CD₃OD 8.77 (s, 1 H), 7.79 (s, 1 H), | 447 | Example |
| | [3-(dimethylamino)propyl]-7- | 7.53 (d, 1 H), 7.45 (d, 1 H), 7.26 (s, | | 15 |
| | methoxyquinoline-3-carboxamide | 1 H), 7.17 (dd, 1 H), 4.00 (s, 3 H), | | |
| | | 3.08 (t, 2 H), 2.79 (s, 6 H), 2.64 (t, | | |
| | | 2 H), 1.84 (m, 2 H) | | |

### Example 14

### 4-[(2,4-Difluorophenyl)amino]-6-(3-hydroxypropyl)-7-methoxyquinoline-3-carboxamide

A solution of 6-(3-{[tert-butyl(dimethyl)silyl]oxy}propyl)-4-[(2,4-difluorophenyl)amino]-7-methoxyquinoline-3-carboxamide (Intermediate 75, 350 mg, 0.7 mmol) in TBAF (1.0 M in THF, 3.6 mL) was stirred for 16 hours, poured into water (300 mL) and extracted with EtOAc (3×200 mL). The combined organic extracts were dried (Na₂SO₄), filtered, and concentrated under reduced pressure to give 250 mg of an oil. ¹H NMR: 10.70 (s, 1H), 8.92 (s, 1H), 8.27 (s, 1H), 7.64 (s, 1H), 7.33 (m, 2H), 7.28 (s, 1H), 6.98 (m, 2H), 4.37 (t, 1H), 3.92 (s, 3H), 3.29 (m, 2H), 2.51 (t, 2H), 1.46 (m, 2H).

### Examples 15-16

The following compounds were prepared by a similar method to Example 14 using the appropriate starting materials.

| Ex | Name | NMR | MS | SM |
|---|---|---|---|---|
| 15 | 4-[(3,4- | 10.07 (s, 1 H), 8.85 (s, 1 H), 8.11 (s, | 419 | Intermediate |
| | Dichlorophenyl)amino]-6- | 1 H), 7.61 (m, 2 H), 7.43 (d, 1 H), | | 76 |
| | (3-hydroxypropyl)-7- | 7.33 (s, 1 H), 7.13 (s, 1 H), 6.86 (d, | | |
| | methoxyquinoline-3- | 1 H), 4.43 (t, 1 H), 3.95 (s, 3 H), | | |
| | carboxamide | 3.16 (m, 2 H), 2.62 (m, 2 H), 1.59 | | |
| | | (m, 2 H) | | |
| 16 | 4-[(2,4- | CD₃OD 8.71 (s, 1 H), 7.36 (s, 1 H), | 402 | Intermediate |
| | Difluorophenyl)amino]-7- | 7.11 (s, 1 H), 6.97 (m, 2 H), 6.81 | | 77 |
| | ethoxy-6-(3- | (m, 1 H), 4.11 (q, 2 H), 3.36 (t, 2 | | |
| | hydroxypropyl)quinoline- | H), 2.50 (t, 2 H), 1.55 (m, 2 H), | | |
| | 3-carboxamide | 1.38 (t, 3 H) | | |

### Examples 17-19

The following compounds were prepared by methods similar to those for Example 2 and Example 14, but without isolating the TBDMS ether amide prior to the deprotection step.

| Ex | Name | NMR | MS | SM |
|---|---|---|---|---|
| 17 | 4-[(3-Chloro-4- | 10.25 (s, 1 H), 8.87 (s, 1 H), 8.15 | 404 | Intermediate |
| | fluorophenyl)amino]-6-(3- | (s, 1 H), 7.60 (s, 1 H), 7.50 (s, 1 | | 66 |
| | hydroxypropyl)-7- | H), 7.31 (s, 1 H), 7.26 (t, 1 H), | | |
| | methoxyquinoline-3- | 7.12 (d, 1 H), 6.89 (m, 1 H), 4.43 | | |
| | carboxamide | (t, 1 H), 3.94 (s, 3 H), 3.35 (m, 2 | | |
| | | H), 2.59 (m, 2 H), 1.54 (m, 2 H) | | |
| 18 | 4-[(3-Chloro-2- | 10.55 (s, 1 H), 8.87 (s, 1 H), 8.23 | 404 | Intermediate |
| | fluorophenyl)amino]-6-(3- | (s, 1 H), 7.61 (s, 1 H), 7.33 (s, 1 | | 67 |
| | hydroxypropyl)-7- | H), 7.25 (s, 1 H), 7.10 (m, 1 H), | | |
| | methoxyquinoline-3- | 6.92 (t, 1 H), 6.69 (t, 1 H), 4.31 | | |
| | carboxamide | (t, 1 H), 3.84 (s, 3 H), 3.25 (m, 2 | | |
| | | H), 2.48 (m, 2 H), 1.41 (m, 2 H) | | |
| 19 | 4-[(2,3- | 10.74 (s, 1 H), 8.96 (s, 1 H), 8.34 | 420 | Intermediate |
| | Dichlorophenyl)amino]-6-(3- | (s, 1 H), 7.74 (s, 1 H), 7.30 (s, 1 | | 65 |
| | hydroxypropyl)-7- | H), 7.21 (m, 2 H), 7.05 (t, 1 H), | | |
| | methoxyquinoline-3- | 6.53 (d, 1 H), 4.33 (t, 1 H), 3.90 | | |
| | carboxamide | (s, 3 H), 3.23 (m, 2 H), 2.50 (m, 2 | | |
| | | H), 1.43 (m, 2 H) | | |

### Example 20

### 4-[(4-Ethylphenyl)amino]-6-(3-hydroxypropyl)-7-methoxyquinoline-3-carboxamide

A solution of 6-(3-{[tert-butyl(dimethyl)silyl]oxy}prop-1-yn-1-yl)-4-[(4-ethylphenyl)amino]-7-methoxyquinoline-3-carboxamide (Intermediate 78, 0.30 g, 0.61 mmol) and TBAF (1.0 M in THF, 5 mL, 5 mmol) was stirred for 16 hours. The reaction mixture was partitioned between EtOAc (10 mL) and NaHCO₃ solution (25 mL), and the organic layer extracted and transferred to a pressure bottle with 10% palladium on carbon (30 mg). This was charged with 50 psi of H₂ gas and shaken for 1 hour at 25° C. The resulting black mixture was filtered through diatomaceous earth, concentrated onto silica, and purified by column chromatography (9:1 EtOAc:MeOH), to give 78 mg (33%) of a light yellow solid. ¹H NMR: 10.76 (s, 1H), 8.90 (s, 1H), 8.21 (s, 1H), 7.59 (s, 1H), 7.33 (s, 1H), 7.24 (s, 1H), 7.09 (d, 2H), 6.89 (d, 2H), 4.35 (t, 1H), 3.91 (s, 3H), 3.26 (m, 2H), 2.59 (m, 4H), 1.39 (m, 2H), 1.15 (t, 3H); m/z: 380.

### Example 21

### 4-[(4-Ethylphenyl)amino]-6-(3-hydroxyprop-1-yn-1-yl)-7-methoxyquinoline-3-carboxamide

A solution of 6-(3-{[tert-butyl(dimethyl)silyl]oxy}prop-1-yn-1-yl)-4-[(4-ethylphenyl)amino]-7-methoxyquinoline-3-carboxamide (Intermediate 78, 200 mg, 0.41 mmol) and TBAF (1.0 M in THF, 5 mL, 5 mmol) was stirred for 16 hours. The reaction mixture was partitioned between EtOAc (25 mL) and NaHCO₃ soln (25 mL), and the organic layer was extracted, concentrated under reduced pressure, and the residue purified by column chromatography (hexanes/EtOAc) to give 33 mg (21%) of a yellow solid. ¹H NMR: 10.62 (s, 1H), 8.91 (s, 1H), 8.20 (s, 1H), 7.72 (s, 1H), 7.61 (s, 1H), 7.31 (s, 1H), 7.13 (d, 2H), 6.91 (d, 2H), 5.29 (t, 1H), 4.23 (d, 2H), 3.93 (s, 3H), 2.55 (m, 2H), 1.15 (t, 3H); m/z: 376.

### Example 22

### 7-Ethoxy-4-[(4-ethylphenyl)amino]-6-(3-hydroxypropyl)quinoline-3-carboxamide

A solution of 7-ethoxy-4-[(4-ethylphenyl)amino]-6-[3-(tetrahydro-2H-pyran-2-yloxy)propyl]quinoline-3-carboxamide (Example 4; 122 mg, 0.31 mmol), 4 N HCl in dioxane (2 mL), and MeOH (10 mL) was allowed to stand for 3 days. The solvent was removed under reduced pressure and the residue partitioned between 1 N NaOH (15 mL) and EtOAc (15 mL). The aqueous layer was further extracted with EtOAc (3×20 mL), and the combined organic extracts were dried (Na₂SO₄), filtered, and concentrated under reduced pressure. The residue was purified by chromatography (EtOAc/MeOH), to give 25 mg of a gum. ¹H NMR (CD₃OD): 8.69 (s, 1H), 7.32 (s, 1H), 7.06 (d, 2H), 7.04 (s, 1H), 6.85 (d, 2H), 4.08 (q, 2H), 3.31 (t, 2H), 2.53 (m, 2H), 2.42 (t, 2H), 1.46 (m, 2H), 1.37 (t, 3H), 1.13 (t, 3H); m/z: 394.

### Examples 23-25

The following compounds were prepared by a method similar to Example 22 using the appropriate starting materials.

| Ex | Name | NMR | MS | SM |
|---|---|---|---|---|
| 23 | 4-[(3,4- | CD₃OD 8.84 (s, 1H), 7.65 (s, 1H), | 434 | Example 5 |
| | Dichlorophenyl)amino]-7- | 7.40 (d, 1H), 7.30 (s, 1H), 7.14 (d, 1H), | | |
| | ethoxy-6-(3- | 6.92 (dd, 1H), 4.25 (q, 2H), 3.53 (t, 2H), | | |
| | hydroxypropyl)quinoline-3- | 2.72 (t, 2H), 1.75 (m, 2H), 1.53 (t, 3H) | | |
| | carboxamide | | | |
| 24 | 4-[(2,3- | 10.85 (s, 1H), 9.07 (s, 1H), 8.45 (s, 1H), | 434 | Example 6 |
| | Dichlorophenyl)amino]-7- | 7.84 (s, 1H), 7.39 (s, 1H), 7.31 (m, 2H), | | |
| | ethoxy-6-(3- | 7.16 (t, 1H), 6.63 (d, 1H), 4.43 (t, 1H), | | |
| | hydroxypropyl)quinoline-3- | 4.28 (q, 2H), 3.35 (m, 2H), 2.61 (t, 2H), | | |
| | carboxamide | 1.57 (m, 2H), 1.48 (t, 3H) | | |
| 25 | 4-[(3-Chloro-4- | CD₃OD 8.70 (s, 1H), 7.45 (s, 1H), | 418 | Example 7 |
| | fluorophenyl)amino]-7- | 7.14 (s, 1H), 7.04 (m, 1H), 6.98 (m, 1H), | | |
| | ethoxy-6-(3- | 6.85 (d, 1H), 4.11 (q, 2H), 3.39 (t, 2H), | | |
| | hydroxypropyl)quinoline-3- | 2.56 (t, 2H), 1.60 (m, 2H), 1.40 (t, 3H) | | |
| | carboxamide | | | |

### Example 26

### 6-Cyclopropyl-4-[(3,4-dichlorophenyl)amino]-7-methoxyquinoline-3-carboxamide

A mixture of 6-bromo-4-[(3,4-dichlorophenyl)amino]-7-methoxyquinoline-3-carboxamide (Intermediate 70; 297 mg, 0.62 mmol), cyclopropyl boronic acid (127 mg, 1.5 mmol), tetrakis(triphenylphosphine)palladium (0) (180 mg, 0.155 mmol), potassium phosphate (829 mg, 4 mmol) and a toluene/water mixture (10 mL, 20:1) was stirred for 16 hours at 100° C. The reaction mixture was filtered through diatomaceous earth, and washed with EtOAc. The organic layer was washed with water and brine, dried (Na₂SO₄), and concentrated under reduced pressure. The residue was purified first with Berger supercritical fluid chromatography, and then by reverse phase chromatography on the Gilson to give 12 mg of product. ¹H NMR (CD₃OD): 8.82 (s, 1H), 7.62 (d, 1H), 7.49 (d, 1H), 7.37 (s, 1H), 7.26 (d, 1H), 7.24 (s, 1H), 4.08 (s, 3H), 2.18 (m, 1H), 0.94 (m, 2H), 0.38 (m, 2H); m/z: 402.

### Examples 27-30

The following compounds were prepared by a similar method to Example 26 using the appropriate starting materials.

| Ex | Name | NMR | MS | SM |
|---|---|---|---|---|
| 27 | 6-Cyclopropyl-4-[(2,4- | CD₃OD 8.83 (s, 1H), 7.50 (m, 1H), | 370 | Intermediate |
| | difluorophenyl)amino]-7- | 7.33 (s, 1H), 7.22 (m, 2H), 7.14 (m, | | 71 |
| | methoxyquinoline-3- | 1H), 4.07 (s, 3H), 2.15 (m, 1H), | | |
| | carboxamide | 0.92 (m, 2H), 0.31 (m, 2H) | | |
| 28 | 7-Cyclopropyl-4-[(2,4- | CD₃OD 8.77 (s, 1H), 7.52 (m, 1H), | 370 | Intermediate |
| | difluorophenyl)amino]-6- | 7.32 (d, 2H), 7.21 (m, 1H), 7.13 (m, | | 72 |
| | methoxyquinoline-3- | 1H), 3.69 (s, 3H), 2.39 (m, 1H), | | |
| | carboxamide | 1.19 (m, 2H), 0.88 (m, 2H) | | |
| 29 | 7-Cyclopropyl-4-[(3,4- | CD₃OD 8.71 (s, 1H), 7.60 (d, 1H), | 402 | Intermediate |
| | dichlorophenyl)amino]-6- | 7.52 (d, 1H), 7.41 (s, 1H), 7.34 (s, 1H), | | 73 |
| | methoxyquinoline-3- | 7.26 (dd, 1H), 3.79 (s, 3H), | | |
| | carboxamide | 2.40 (m, 1H), 1.19 (m, 2H), | | |
| | | 0.89 (m, 2H) | | |
| 30 | 4-[(2,4- | 10.72 (s, 1H), 8.94 (s, 1H), 8.27 (s, | 451 | Intermediate |
| | Difluorophenyl)amino]-7- | 1H), 7.98 (s, 1H), 7.69 (m, 2H), | | 74 |
| | ethoxy-6-(6-methoxypyridin- | 7.55 (s, 1H), 7.40 (s, 1H), 7.38 (m, | | |
| | 3-yl)quinoline-3- | 1H), 7.12 (m, 1H), 7.03 (m, 1H), | | |
| | carboxamide | 6.80 (d, 1H), 4.21 (q, 2H), 3.85 (s, | | |
| | | 3H), 1.34 (t, 3H) | | |

### Example 31

### 4-[(2,4-Difluorophenyl)amino]-7-ethoxy-6-pyridin-3-ylquinoline-3-carboxamide

A mixture of 6-bromo-4-[(2,4-difluorophenyl)amino]-7-ethoxyquinoline-3-carboxamide (Intermediate 74; 100 mg, 0.237 mmol), 3-pyridine boronic acid (35 mg, 0.28 mmol), caesium carbonate (154 mg, 0.47 mmol), tetrakis(triphenylphosphine)palladium (0) (27 mg, 10% mol) in dioxane/water (4 ml, 4:1) was heated under microwave conditions at 165° C. for 30 minutes. The reaction mixture was purified by column chromatography (EtOAc, EtOAc:MeOH 70:30) and recrystallized from MeOH to give 63 mg (63%) of a white solid. ¹H NMR: 10.80 (s, 1H), 9.00 (s, 1H), 8.57 (s, 1H), 8.45 (s, 1H), 8.30 (s, 1H), 7.80 (m, 1H), 7.70 (s, 1H), 7.65 (s, 1H), 7.45 (m, 3H), 7.25 (m, 1H), 7.10 (m, 1H), 4.29 (q, 2H), 1.39 (t, 3H); m/z: 421.

### Examples 32-37

The following compounds were prepared by a similar method to Example 31 from Intermediate 74 and the appropriate boronic acid.

| Ex | Name | NMR | MS |
|---|---|---|---|
| 32 | 4-[(2,4- | 10.66 (m, 1H), 8.98 (s, 1H), 8.31 (s, 1H), | 439 |
| | Difluorophenyl)amino]-6- | 7.69 (s, 1H), 7.42 (s, 2H), 7.34 (m, 1H), 7.11 (m, 1H), | |
| | (3,5-dimethylisoxazol-4-yl)-7- | 6.99 (m, 1H), 4.31 (q, 2H), 2.29 (s, 3H), | |
| | ethoxyquinoline-3- | 2.10 (s, 3H), 1.45 (t, 3H) | |
| | carboxamide | | |
| 33 | 4-[(2,4- | 10.80 (s, 1H), 10.60 (s, 1H), 8.89 (s, 1H), | 409 |
| | Difluorophenyl)amino]-7- | 8.25 (s, 1H), 7.80 (s, 1H), 7.62 (s, 1H), 7.25 (m, 2H), | |
| | ethoxy-6-(1H-pyrrol-2- | 7.00 (m, 2H), 6.83 (s, 1H), 6.06 (m, 2H), | |
| | yl)quinoline-3-carboxamide | 4.30 (q, 2H), 1.50 (t, 3H) | |
| 34 | 4-[(2,4- | 10.69 (s, 1H), 9.12 (s, 1H), 8.95 (s, 1H), | 422 |
| | Difluorophenyl)amino]-7- | 8.75 (s, 2H), 8.25 (s, 1H), 7.75 (s, 1H), 7.63 (s, 1H), | |
| | ethoxy-6-pyrimidin-5- | 7.47 (s, 1H), 7.36 (m, 1H), 7.20 (m, 1H), | |
| | ylquinoline-3-carboxamide | 7.05 (m, 1H), 4.25 (q, 2H), 1.36 (t, 3H) | |
| 35 | 4-[(2,4- | 10.75 (s, 1H), 8.95 (s, 1H), 8.63 (s, 2H), | 421 |
| | Difluorophenyl)amino]-7- | 8.25 (s, 1H), 7.65 (s, 2H), 7.43 (s, 1H), 7.37 (m, 1H), | |
| | ethoxy-6-pyridin-4- | 7.28 (m, 2H), 7.20 (m, 1H), 7.05 (m, 1H), | |
| | ylquinoline-3-carboxamide | 4.25 (q, 2H), 1.35 (t, 3H) | |
| 36 | 4-[(2,4- | 14.80 (br s, 1H), 11.60 (s, 1H), 8.85 (s, 1H), | 410 |
| | Difluorophenyl)amino]-7- | 8.45 (s, 1H), 8.28 (s, 1H), 8.00 (s, 2H), 7.75 (s, | |
| | ethoxy-6-(1H-pyrazol-4- | 1H), 7.50 (m, 3H), 7.20 (s, 1H), 4.30 (q, 2H), | |
| | yl)quinoline-3-carboxamide | 1.52 (t, 3H) | |
| 37 | 4-[(2,4- | 10.75 (s, 1H), 8.88 (s, 1H), 8.23 (s, 1H), | 466 |
| | Difluorophenyl)amino]-7- | 7.86 (s, 1H), 7.76 (s, 1H), 7.60 (s, 1H), 7.40 (m, 1H), | |
| | ethoxy-6-(1-isobutyl-1H- | 7.35 (m, 2H), 7.20 (m, 1H), 7.07 (m, 1H), | |
| | pyrazol-4-yl)quinoline-3- | 4.25 (q, 2H), 3.93 (d, 2H), 2.05 (m, 1H), | |
| | carboxamide | 1.45 (t, 3H), 0.80 (d, 6H) | |

### Example 38

### tert-Butyl 4-{3-(aminocarbonyl)-4-[(2,4-difluorophenyl)amino]-7-ethoxyquinolin-6-yl}-3,6-dihydropyridine-1(2H)-carboxylate

A mixture of 6-bromo-4-[(2,4-difluorophenyl)amino]-7-ethoxyquinoline-3-carboxamide (Intermediate 74; 0.63 g, 1.50 mmol), tert-butyl 4-(4,4,5,5-tetramethyl-1,3,2-dioxaborolan-2-yl)-3,6-dihydropyridine-1(2H)-carboxylate (0.69 g, 2.25 mmol), Pd(PPh₃)₄ (0.35 g, 0.30 mmol), and potassium carbonate (0.52 g, 3.75 mmol) in dioxane (15 mL) and water (1 mL) under argon was heated to 100° C. for 6 hours. After cooling, the reaction mixture was diluted with water (˜100 mL), and extracted with EtOAc. The combined organic extract was dried (MgSO₄), filtered, and the crude product purified by column chromatography (EtOAc:MeOH, 4:1) to give 622 mg (79%) of an off white solid. ¹H NMR: 10.76 (s, 1H), 8.90 (s, 1H), 8.25 (s, 1H), 7.64 (s, 1H), 7.37 (m, 2H), 7.27 (s, 1H), 7.06 (m, 2H), 5.52 (s, 1H), 4.16 (q, 2H), 3.85 (s, 2H), 3.39 (s, 2H), 2.27 (s, 2H), 1.38 (m, 12H); m/z 525.

### Example 39

### 4-[(2,4-Difluorophenyl)amino]-7-ethoxy-6-(1,2,3,6-tetrahydropyridin-4-yl)quinoline-3-carboxamide

A solution of tert-butyl 4-{3-(aminocarbonyl)-4-[(2,4-difluorophenyl)amino]-7-ethoxyquinolin-6-yl}-3,6-dihydropyridine-1(2H)-carboxylate (Example 38, 0.21 g, 0.40 mmol) in CH₂Cl₂ (4 mL) and TFA (4 mL) was stirred for 2 hours, then concentrated under reduced pressure. The residue was dissolved in EtOAc (50 mL) and washed with saturated aqueous NaHCO₃ (50 mL). The organic extract was dried (MgSO₄), filtered and concentrated under reduced pressure, and the residue purified via reverse phase HPLC (acetonitrile/water) to give 161 mg (95%) of the title compound. ¹H NMR 11.41 (s, 1H), 8.89 (s, 1H), 8.26 (s, 1H), 7.77 (s, 2H), 7.46 (m, 1H), 7.38 (m, 2H), 7.14 (m, 1H), 5.76 (s, 1H), 4.25 (q, 2H), 3.70 (s, 2H), 3.22 (s, 2H), 2.53 (s, 2H), 1.42 (t, 3H); m/z 427.

### Example 40

### 4-[(2,4-Difluorophenyl)amino]-7-ethoxy-6-piperidin-4-ylquinoline-3-carboxamide

A solution of 4-[(2,4-difluorophenyl)amino]-7-ethoxy-6-(1,2,3,6-tetrahydropyridin-4-yl)quinoline-3-carboxamide (Example 39, 0.201 g, 0.473 mmol), TFA (2 mL) and triethylsilane (1 mL) was warmed to 50° C. for 24 hours before being concentrated under reduced pressure. The residue was purified via reverse phase HPLC (acetonitrile/water) to give 15 mg (7.5%) of the title compound. m/z 427.

### Example 41

### 6-(1-Acetyl-1,2,3,6-tetrahydropyridin-4-yl)-4-[(2,4-difluorophenyl)amino]-7-ethoxyquinoline-3-carboxamide

A solution of 4-[(2,4-difluorophenyl)amino]-7-ethoxy-6-(1,2,3,6-tetrahydropyridin-4-yl)quinoline-3-carboxamide (Example 39, 0.15 g, 0.353 mmol) and triethylamine (0.15 mL, 1.06 mmol) in CH₂Cl₂ was cooled to 0° C. and acetic anhydride (0.054 g, 0.529 mmol) was added dropwise. The reaction was warmed to room temperature and stirred for 12 hours, then added to aqueous NaHCO₃ (25 mL) and extracted with EtOAc (2×25 ml). The combined organic extract was dried (MgSO₄), filtered and concentrated under reduced pressure, and the residue purified via reverse phase HPLC (acetonitrile/water) to give 100 mg (61%) of the title compound. ¹H NMR 10.75 (s, 1H), 8.90 (s, 1H), 8.26 (s, 1H), 7.65 (s, 1H), 7.38 (m, 2H), 7.28 (s, 1H), 7.04 (m, 2H), 5.50 (d, 1H), 4.18 (q, 2H), 3.92 (d, 2H), 3.50 (m, 2H), 2.35 (s, 1H), 2.25 (s, 1H), 2.02 (s, 3H), 1.38 (t, 3H); m/z 467.

### Example 42

### 7-Ethoxy-4-[(2-fluoro-4-methylphenyl)amino]-6-(1-methylpiperidin-4-yl)quinoline-3-carboxamide

To a solution of ethyl 7-ethoxy-4-[(2-fluoro-4-methylphenyl)amino]-6-(1-methylpiperidin-4-yl)quinoline-3-carboxylate (Intermediate 15, 100 mg, 0.20 mmol) and formamide (0.100 mL) in THF (5 mL) was added a solution of NaOMe (0.40 mL, 0.5M in MeOH). The reaction was heated to reflux for 3 hours, cooled and concentrated under reduced pressure. The residue was purified by reverse phase HPLC (acetonitrile/water) to give 70 mg (74%) of the title compound. ¹H NMR 12.06 (s, 1H), 8.94 (s, 1H), 8.38 (s, 1H), 7.92 (s, 1H), 7.61 (s, 1H), 7.38 (s, 1H), 7.34 (m, 1H), 7.22 (d, 1H), 7.12 (d, 1H), 4.25 (q, 2H), 3.41 (d, 2H), 3.06 (m, 3H), 2.78 (s, 3H), 2.38 (s, 3H), 1.78 (d, 2H), 1.44 (t, 5H); m/z 437.

### Example 43-58

The following compounds were prepared by a similar method to Example 42.

| Ex | Name | NMR | MS | SM |
|---|---|---|---|---|
| 43 | 4-[(2,4- | MeOD 8.82 (s, 1H), 7.45 (s, 1H), | 441 | Intermediate |
| | Difluorophenyl)amino]-7- | 7.19 (s, 1H), 7.02 (m, 2H), | | 16 |
| | ethoxy-6-(1- | 6.87 (m, 1H), 4.18 (q, 2H), 2.86 (m, 3H), | | |
| | methylpiperidin-4- | 2.26 (s, 3H), 2.10 (m, 2H), | | |
| | yl)quinoline-3- | 1.68 (d, 2H), 1.46 (t, 3H), 1.34 (m, | | |
| | carboxamide | 2H) | | |
| 44 | 4-[(3-Chloro-2- | 11.55 (s, 1H), 8.92 (s, 1H), 8.29 (s, | 457 | Intermediate |
| | fluorophenyl)amino]-7- | 1H), 7.81 (s, 2H), 7.48 (m, 1H), | | 17 |
| | ethoxy-6-(1- | 7.43 (s, 1H), 7.26 (m, 2H), 4.27 (q, | | |
| | methylpiperidin-4- | 2H), 3.45 (d, 2H), 3.10 (m, 3H), | | |
| | yl)quinoline-3- | 2.79 (s, 3H), 1.89 (d, 2H), 1.61 (m, | | |
| | carboxamide | 2H), 1.45 (t, 3H) | | |
| 45 | 4-[(2,3- | 10.44 (s, 1H), 9.10 (s, 1H), 8.52 (s, | 473 | Intermediate |
| | Dichlorophenyl)amino]-7- | 1H), 7.92 (s, 1H), 7.62 (d, 1H), | | 18 |
| | ethoxy-6-(1- | 7.54 (s, 2H), 7.41 (m, 2H), 4.25 (q, | | |
| | methylpiperidin-4- | 2H), 3.37 (d, 2H), 3.04 (m, 3H), | | |
| | yl)quinoline-3- | 2.72 (s, 3H), 1.78 (m, 2H), | | |
| | carboxamide | 1.55 (m, 2H), 1.45 (t, 3H) | | |
| 46 | 4-[(2,4- | 10.77 (s, 1H), 8.91 (s, 1H), 8.28 (s, | 469 | Intermediate |
| | Difluorophenyl)amino]-7- | 1H), 7.65 (s, 1H), 7.36 (m, 2H), | | 19 |
| | ethoxy-6-(1- | 7.26 (s, 1H), 7.02 (m, 2H), 4.19 (q, | | |
| | isopropylpiperidin-4- | 2H), 2.94 (m, 4H), 2.43 (m, 2H), | | |
| | yl)quinoline-3- | 1.64 (d, 2H), 1.40 (t, 3H), 1.26 (m, | | |
| | carboxamide | 2H), 1.04 (d, 6H) | | |
| 47 | 7-Ethoxy-4-[(2-fluoro-4- | 10.93 (s, 1H), 8.90 (s, 1H), 8.27 (s, | 465 | Intermediate |
| | methylphenyl)amino]-6- | 1H), 7.62 (s, 1H), 7.32 (s, 1H), | | 20 |
| | (1-isopropylpiperidin-4- | 7.22 (s, 1H), 7.12 (d, 1H), 6.92 (d, | | |
| | yl)quinoline-3- | 2H), 4.17 (q, 2H), 2.81 (m, 4H), | | |
| | carboxamide | 2.29 (m, 5H), 1.55 (d, 2H), 1.38 (t, | | |
| | | 3H), 1.12 (m, 2H), 1.00 (d, 6H) | | |
| 48 | 7-Ethoxy-4-[(2-fluoro-5- | 10.85 (s, 1H), 8.93 (s, 1H), 8.31 (s, | 465 | Intermediate |
| | methylphenyl)amino]-6- | 1H), 7.67 (s, 1H), 7.41 (s, 1H), | | 21 |
| | (1-isopropylpiperidin-4- | 7.26 (s, 1H), 7.15 (dd, 1H), | | |
| | yl)quinoline-3- | 6.86 (m, 1H), 6.73 (d, 1H), 4.20 (q, 2H), | | |
| | carboxamide | 2.92 (m, 4H), 2.45 (m, 2H), | | |
| | | 2.13 (s, 3H), 1.64 (d, 2H), 1.40 (t, | | |
| | | 3H), 1.25 (m, 2H), 1.03 (d, 6H) | | |
| 49 | 4-[(2-Fluoro-4- | 10.95 (s, 1H), 8.93 (s, 1H), 8.29 (s, | 423 | Intermediate |
| | methylphenyl)amino]-7- | 1H), 7.66 (s, 1H), 7.35 (s, 1H), | | 22 |
| | methoxy-6-(1- | 7.27 (s, 1H), 7.13 (m, 1H), | | |
| | methylpiperidin-4- | 6.95 (m, 2H), 3.95 (s, 3H), 2.71 (m, 3H), | | |
| | yl)quinoline-3- | 2.30 (s, 3H), 2.15 (s, 3H), | | |
| | carboxamide | 1.88 (m, 2H), 1.49 (m, 2H), 1.11 (m, 2H) | | |
| 50 | 4-[(3-Chloro-2- | 10.80 (s, 1H), 9.14 (s, 1H), 8.37 (s, | 443 | Intermediate |
| | fluorophenyl)amino]-7- | 1H), 7.78 (s, 1H), 7.39 (s, 2H), | | 23 |
| | methoxy-6-(1- | 7.24 (m, 1H), 7.06 (m, 1H), | | |
| | methylpiperidin-4- | 6.80 (m, 1H), 3.98 (s, 3H), 3.03 (m, 3H), | | |
| | yl)quinoline-3- | 2.70 (s, 3H), 2.48 (m, 2H), | | |
| | carboxamide | 1.80 (m, 2H), 1.45 (m, 2H) | | |
| 51 | 4-[(2,4- | 10.85 (s, 1H), 8.93 (s, 1H), 8.30 (s, | 427 | Intermediate |
| | Difluorophenyl)amino]-7- | 1H), 7.67 (s, 1H), 7.42 (m, 1H), | | 24 |
| | methoxy-6-(1- | 7.34 (s, 1H), 7.29 (s, 1H), 7.07 (m, | | |
| | methylpiperidin-4- | 2H), 3.94 (s, 3H), 2.74 (m, 3H), | | |
| | yl)quinoline-3- | 2.13 (s, 3H), 1.88 (m, 2H), | | |
| | carboxamide | 1.55 (m, 2H), 1.16 (m, 2H) | | |
| 52 | 4-[(2-Fluoro-4- | 10.96 (s, 1H), 8.93 (s, 1H), 8.29 (s, | 451 | Intermediate |
| | methylphenyl)amino]-6- | 1H), 7.64 (s, 1H), 7.36 (s, 1H), | | 25 |
| | (1-isopropylpiperidin-4- | 7.25 (s, 1H), 7.13 (d, 1H), 6.96 (s, | | |
| | yl)-7-methoxyquinoline-3- | 2H), 3.92 (s, 3H), 2.74 (m, 4H), | | |
| | carboxamide | 2.31 (s, 3H), 2.12 (m, 2H), | | |
| | | 1.54 (m, 2H), 1.10 (m, 2H), 0.93 (d, 6H) | | |
| 53 | 4-[(2,4- | CD₂Cl₂ 10.51 (s, 1H), 8.79 (s, 1H), | 455 | Intermediate |
| | Difluorophenyl)amino]-6- | 7.42 (s, 1H), 7.28 (s, 1H), 6.98 (m, | | 26 |
| | (1-isopropylpiperidin-4- | 2H), 6.83 (m, 1H), 5.98 (br s, 2H), | | |
| | yl)-7-methoxyquinoline-3- | 3.95 (s, 3H), 3.03 (m, 2H), | | |
| | carboxamide | 2.87 (m, 2H), 2.41 (m, 2H), 1.67 (m, 2H), | | |
| | | 1.45 (m, 2H), 1.11 (d, 6H) | | |
| 54 | 4-[(3-Chloro-2- | CD₂Cl₂ 10.49 (s, 1H), 8.85 (s, 1H), | 471 | Intermediate |
| | fluorophenyl)amino]-6-(1- | 7.50 (s, 1H), 7.34 (s, 1H), 7.12 (m, | | 27 |
| | isopropylpiperidin-4-yl)-7- | 1H), 6.96 (m, 1H), 6.81 (m, 1H), | | |
| | methoxyquinoline-3- | 6.13 (br s, 2H), 3.97 (s, 3H), | | |
| | carboxamide | 3.07 (m, 3H), 2.93 (m, 1H), 2.41 (m, 2H), | | |
| | | 1.71 (m, 2H), 1.54 (m, 2H), | | |
| | | 1.13 (d, 6H) | | |
| 55 | 6-(1-Acetylpiperidin-4- | 10.94 (s, 1H), 8.90 (s, 1H), 8.25 (s, | 455 | Intermediate |
| | yl)-4-[(2,4- | 1H), 7.68 (s, 1H), 7.45 (s, 1H), | | 79 |
| | difluorophenyl)amino]-7- | 7.38 (m, 1H), 7.29 (s, 1H), | | |
| | methoxyquinoline-3- | 7.21 (m, 1H), 7.04 (m, 1H), 4.43 (m, 1H), | | |
| | carboxamide | 3.96 (s, 3H), 3.81 (m, 1H), | | |
| | | 3.09 (m, 2H), 2.55 (m, 1H), | | |
| | | 1.98 (s, 3H), 1.64 (m, 2H), 1.05 (m, 2H) | | |
| 56 | 6-(1-Acetylpiperidin-4- | 11.10 (s, 1H), 8.92 (s, 1H), 8.30 (s, | 465 | Intermediate |
| | yl)-7-ethoxy-4-[(2-fluoro- | 1H), 7.67 (s, 1H), 7.30 (s, 1H), | | 80 |
| | 4- | 7.24 (s, 1H), 7.14 (m, 1H), | | |
| | methylphenyl)amino]quinoline- | 6.98 (m, 2H), 4.40 (m, 1H), 4.20 (m, 2H), | | |
| | 3-carboxamide | 3.79 (m, 1H), 3.04 (m, 2H), | | |
| | | 2.28 (s, 3H), 1.98 (s, 3H), 1.60 (m, | | |
| | | 2H), 1.39 (m, 3H), 0.89 (m, 2H), | | |
| | | One proton masked by solvent | | |
| 57 | 7-Ethoxy-4-[(2-fluoro-4- | CD₂Cl₂ 10.63 (s, 1H), 8.75 (s, 1H), | 509 | Intermediate |
| | methylphenyl)amino]-6- | 7.32 (s, 1H), 7.23 (s, 1H), 6.93 (m, | | 81 |
| | [1-(3- | 2H), 6.88 (m, 1H), 5.94 (br s, 2H), | | |
| | methoxypropanoyl)piperidin- | 4.58 (m, 1H), 4.19 (q, 2H), | | |
| | 4-yl]quinoline-3- | 3.82 (m, 1H), 3.65 (m, 2H), 3.33 (s, 3H), | | |
| | carboxamide | 3.04 (m, 2H), 2.54 (m, 3H), | | |
| | | 2.33 (s, 3H), 1.64 (m, 2H), 1.44 (t, | | |
| | | 3H), 1.01 (m, 2H) | | |
| 58 | 7-Ethoxy-4-[(2-fluoro-4- | 11.00 (s, 1H), 8.94 (s, 1H), 8.29 (s, | 495 | Intermediate |
| | methylphenyl)amino]-6- | 1H), 7.65 (s, 1H), 7.34 (s, 1H), | | 82 |
| | {1-[(2R)-2- | 7.22 (s, 1H), 7.11 (m, 1H), | | |
| | hydroxypropanoyl]piperidin- | 6.95 (m, 2H), 4.77 (m, 1H), 4.40 (m, 2H), | | |
| | 4-yl}quinoline-3- | 4.21 (q, 2H), 3.95 (m, 1H), | | |
| | carboxamide | 3.03 (m, 2H), 2.65 (m, 1H), | | |
| | | 2.28 (s, 3H), 1.62 (m, 2H), 1.41 (t, 3H), | | |
| | | 1.18 (d, 3H), 0.91 (m, 2H) | | |

### Example 59

### 4-[(2,4-Difluorophenyl)amino]-7-ethoxy-6-(6-oxo-1,6-dihydropyridin-3-yl)quinoline-3-carboxamide

To a suspension of 4-(2,4-difluorophenylamino)-7-ethoxy-6-(6-methoxypyridin-3-yl)quinoline-3-carboxamide (Example 30, 260 mg, 0.58 mmol) in acetonitrile (25 mL) at 0° C. was added sodium iodide (0.094 mL, 2.31 mmol) and tert-butyldimethylsilyl chloride (261 mg, 1.73 mmol). The cooling bath was removed, and after two hours stirring at RT gave no reaction, the reaction was heated to 50° C. for 40 hours. Water (10 mL) was added, and the mixture washed with EtOAc (100 mL). The aqueous layer was filtered to give a yellow precipitate, which was washed with water and EtOAc to give 123 mg (49%) of a yellow solid. ¹H NMR 11.94 (s, 1H), 11.19 (s, 1H), 8.82 (s, 1H), 8.15 (s, 2H), 7.60 (s, 1H), 7.57 (m, 2H), 7.41 (m, 3H), 7.15 (m, 1H), 6.39 (d, 1H), 4.25 (q, 2H), 1.41 (t, 3H); m/z 437.

### Example 60

### 4-[(2-Fluoro-4-methylphenyl)amino]-6-[1-(2-hydroxyethyl)piperidin-4-yl]-7-methoxyquinoline-3-carboxamide hydrochloride

A mixture of ethyl 6-(1-(2-(tert-butyldimethylsilyloxy)ethyl)piperidin-4-yl)-4-(2-fluoro-4-methylphenylamino)-7-methoxyquinoline-3-carboxylate (Intermediate 86, 0.216 g, 0.36 mmol), formamide (0.144 mL, 3.62 mmol) in DMF (10 mL) was stirred at 100° C. for 3 hours. A solution of NaOMe (1.09 mL, 0.5M in MeOH, 0.54 mmol) was added, and after 6 hours heating, an additional portion of NaOMe (1.09 mL, 0.5M in MeOH, 0.54 mmol). The reaction mixture was stirred overnight, cooled, and tetrabutylammonium fluoride (0.362 ml, 1.0M in THF, 0.36 mmol) added. The reaction was incomplete after stirring overnight, and additional portions of tetrabutylammonium fluoride were added until no starting material remained. The solvent was removed under reduced pressure and the residue purified by column chromatography (CH₂Cl₂/20% 2N NH₃ in MeOH). The crude product was dissolved in MeOH and converted to the HCl salt with a solution of HCl (4N in dioxane). The solvents were removed, and the residue was triturated with CH₃CN and filtered to give 70 mg solid. ¹H NMR 10.72 (s, 1H), 8.84 (s, 1H), 8.28 (s, 1H), 7.65 (s, 1H), 7.36 (m, 1H), 7.23 (s, 1H), 7.01 (m, 2H), 6.80 (s, 1H), 4.15 (m, 2H), 3.52 (t, 2H), 3.25 (s, 3H), 3.16 (d, 1H), 2.72 (m, 4H), 2.36 (m, 2H), 2.17 (s, 3H), 2.02 (m, 2H), one proton masked by solvent; m/z 453.

### Preparation of Starting Materials

### Intermediate 1

### Ethyl 6-{3-[(tert-butoxycarbonyl)amino]propyl}-4-[(3,4-dichlorophenyl)amino]-7-methoxyquinoline-3-carboxylate

A solution of tert-butyl {3-[(1 s,5s)-9-borabicyclo[3.3.1]non-9-yl]propyl}carbamate (1.9 mmol) in THF (5 mL) was prepared from 9-BBN and tert-butyl allylcarbamate by the method of Suzuki et al (JACS, 1989, 111, 314-321). To this solution under N₂ was added K₂CO₃ (248 mg, 1.8 mmol), DMF (5 mL), [1,1′-bis(diphenylphosphino)ferrocene]-dichloropalladium(II) (117 mg, 0.14 mmol), and ethyl 6-bromo-4-[(3,4-dichlorophenyl)amino]-7-methoxyquinoline-3-carboxylate (Intermediate 2; 400 mg, 0.9 mmol). After 16 hours at 50° C., the reaction mixture was cooled, poured into brine (200 mL) and extracted with EtOAc (3×200 mL). The combined organic extracts were dried (Na₂SO₄), filtered, and concentrated under reduced pressure. The crude oil was subjected to normal phase chromatography on the ISCO eluting with EtOAc/10% MeOH:EtOAc to give 500 mg of an oil. m/z: 519.

### Intermediate 2

### Ethyl-6-bromo-4-[(3,4-dichlorophenyl)amino]-7-methoxyquinoline-3-carboxylate

A mixture of ethyl 6-bromo-4-chloro-7-methoxyquinoline-3-carboxylate (Intermediate 28; 750 mg, 2.18 mmol), 3,4-dichloroaniline (389 mg, 2.4 mmol), and acetic acid (1 mL), in ethanol (50 mL) was heated to reflux for 1.5 hours. After cooling, the mixture was neutralized with 2N aqueous ammonia. The resulting solid was collected, washed with water followed by cold ethanol, and dried to give 700 mg solid. ¹H NMR: 8.89 (s, 1H), 8.46 (s, 1H), 7.50 (m, 2H), 7.24 (d, 1H), 6.96 (dd, 1H), 4.03 (s, 3H), 3.98 (q, 2H), 1.12 (t, 3H); m/z: 470.

### Intermediates 3-27

The following compounds were prepared by a similar method to Intermediate 2 using the appropriate starting materials. In some cases, after cooling and addition of aqueous ammonia, the resulting solution was concentrated and purified with silica chromatography.

| Int | Compound | NMR/M/z | SM |
|---|---|---|---|
| 3 | Ethyl 6-bromo-4-[(2,4- | 435 | Intermediate 28 |
| | difluorophenyl)amino]-7- | | |
| | methoxyquinoline-3-carboxylate | | |
| 4 | Ethyl 6-bromo-4-[(2,4- | 452 | Intermediate |
| | difluorophenyl)amino]-7- | | 35 |
| | ethoxyquinoline-3-carboxylate | | |
| 5 | Ethyl 6-bromo-4-[(4- | 9.79 (s, 1H), 8.86 (s, 1H), | Intermediate |
| | ethylphenyl)amino]-7- | 8.16 (s, 1H), 7.41 (s, 1H), | 28 |
| | methoxyquinoline-3-carboxylate | 7.18 (d, 2H), 7.01 (d, 2H), | |
| | | 3.99 (m, 5H), 2.58 (m, 2H), | |
| | | 1.15 (m, 6H) | |
| 6 | Ethyl 6-bromo-7-ethoxy-4-[(4- | 445 | Intermediate |
| | ethylphenyl)amino]quinoline-3- | | 35 |
| | carboxylate | | |
| 7 | Ethyl 6-bromo-4-[(3,4- | 9.62 (1H, s), 8.88 (1H, s), | Intermediate |
| | dichlorophenyl)amino]-7- | 8.46 (1H, s), 7.50 (1H, d), | 35 |
| | ethoxyquinoline-3-carboxylate | 7.47 (1H, s), 7.24 (1H, d), | |
| | | 6.96 (1H, dd), 4.30 (2H, | |
| | | q), 3.98 (2H, q), 1.45 (3H, | |
| | | t), 1.11 (3H, t) | |
| 8 | Ethyl 6-bromo-4-[(2,3- | 485 | Intermediate |
| | dichlorophenyl)amino]-7- | | 35 |
| | ethoxyquinoline-3-carboxylate | | |
| 9 | Ethyl 6-bromo-4-[(3-chloro-4- | | Intermediate |
| | fluorophenyl)amino]-7- | | 35 |
| | ethoxyquinoline-3-carboxylate | | |
| 10 | Ethyl 6-bromo-4-[(2,3- | 471 | Intermediate |
| | dichlorophenyl)amino]-7- | | 28 |
| | methoxyquinoline-3-carboxylate | | |
| 11 | Ethyl 6-bromo-4-[(3-chloro-4- | 454 | Intermediate |
| | fluorophenyl)amino]-7- | | 28 |
| | methoxyquinoline-3-carboxylate | | |
| 12 | Ethyl 6-bromo-4-[(3-chloro-2- | 453 | Intermediate |
| | fluorophenyl)amino]-7- | | 28 |
| | methoxyquinoline-3-carboxylate | | |
| 13 | Ethyl 7-bromo-4-[(2,4- | 439 | Intermediate |
| | difluorophenyl)amino]-6- | | 31 |
| | methoxyquinoline-3-carboxylate | | |
| 14 | Ethyl 7-bromo-4-[(3,4- | | Intermediate |
| | dichlorophenyl)amino]-6- | | 31 |
| | methoxyquinoline-3-carboxylate | | |
| 15 | Ethyl 7-ethoxy-4-[(2-fluoro-4- | 467 | Intermediate |
| | methylphenyl)amino]-6-(1- | | 37 |
| | methylpiperidin-4-yl)quinoline-3- | | |
| | carboxylate | | |
| 16 | Ethyl 4-[(2,4-difluorophenyl)amino]- | 470 | Intermediate |
| | 7-ethoxy-6-(1-methylpiperidin-4- | | 37 |
| | yl)quinoline-3-carboxylate | | |
| 17 | Ethyl 4-[(3-chloro-2- | 487 | Intermediate |
| | fluorophenyl)amino]-7-ethoxy-6-(1- | | 37 |
| | methylpiperidin-4-yl)quinoline-3- | | |
| | carboxylate | | |
| 18 | Ethyl 4-[(2,3-dichlorophenyl)amino]- | 503 | Intermediate |
| | 7-ethoxy-6-(1-methylpiperidin-4- | | 37 |
| | yl)quinoline-3-carboxylate | | |
| 19 | Ethyl 4-[(2,4-difluorophenyl)amino]- | 499 | Intermediate |
| | 7-ethoxy-6-(1-isopropylpiperidin-4- | | 38 |
| | yl)quinoline-3-carboxylate | | |
| 20 | Ethyl 7-ethoxy-4-[(2-fluoro-4- | 494 | Intermediate |
| | methylphenyl)amino]-6-(1- | | 38 |
| | isopropylpiperidin-4-yl)quinoline-3- | | |
| | carboxylate | | |
| 21 | Ethyl 7-ethoxy-4-[(2-fluoro-5- | 494 | Intermediate |
| | methylphenyl)amino]-6-(1- | | 38 |
| | isopropylpiperidin-4-yl)quinoline-3- | | |
| | carboxylate | | |
| 22 | Ethyl 4-[(2-fluoro-4- | 452 | Intermediate |
| | methylphenyl)amino]-7-methoxy-6- | | 39 |
| | (1-methylpiperidin-4-yl)quinoline-3- | | |
| | carboxylate | | |
| 23 | Ethyl 4-[(3-chloro-2- | 472 | Intermediate |
| | fluorophenyl)amino]-7-methoxy-6- | | 39 |
| | (1-methylpiperidin-4-yl)quinoline-3- | | |
| | carboxylate | | |
| 24 | Ethyl 4-[(2,4-difluorophenyl)amino]- | 456 | Intermediate |
| | 7-methoxy-6-(1-methylpiperidin-4- | | 39 |
| | yl)quinoline-3-carboxylate | | |
| 25 | Ethyl 4-[(2-fluoro-4- | 480 | Intermediate |
| | methylphenyl)amino]-6-(1- | | 40 |
| | isopropylpiperidin-4-yl)-7- | | |
| | methoxyquinoline-3-carboxylate | | |
| 26 | Ethyl 4-[(2,4-difluorophenyl)amino]- | 484 | Intermediate |
| | 6-(1-isopropylpiperidin-4-yl)-7- | | 40 |
| | methoxyquinoline-3-carboxylate | | |
| 27 | Ethyl 4-[(3-chloro-2- | 500 | Intermediate |
| | fluorophenyl)amino]-6-(1- | | 40 |
| | isopropylpiperidin-4-yl)-7- | | |
| | methoxyquinoline-3-carboxylate | | |

### Intermediate 28

### Ethyl 6-bromo-4-chloro-7-methoxyquinoline-3-carboxylate

This compound was described in WO 2002092571, and prepared in accordance with the procedures described in Burke T. R. et al., J. Med. Chem., 36 (1993) 425-432.

A solution of ethyl 6-bromo-7-methoxy-4-oxo-1,4-dihydroquinoline-3-carboxylate

(Intermediate 29; 8.0 g, 0.025) in phosphorous oxychloride (100 mL) was heated under reflux overnight. After cooling, the solution was carefully poured into 400 mL of ice water with stirring. The resulting mixture was made just basic with 2N NaOH and extracted with EtOAc. The organic layer was washed with water, dried (Na₂SO₄), and concentrated under reduced pressure to give 8.0 g (93%) of a white solid. ¹H NMR: 9.14 (s, 1H), 8.55 (s, 1H), 7.66 (s, 1H), 4.42 (d, 2H), 4.09 (s, 3H), 1.38 (t, 3H); m/z: 344.

### Intermediate 29

### Ethyl 6-bromo-7-methoxy-4-oxo-1,4-dihydroquinoline-3-carboxylate

A solution of diethyl {[(4-bromo-3-methoxyphenyl)amino]methylene}malonate (Intermediate 30; 11 g, 0.029 mol) in warm diphenyl ether (20 mL) was added dropwise over 15 minutes to refluxing diphenyl ether (180 mL). After 3 hours, the solution was cooled, diluted with hexane (200 mL), and the resulting precipitate collected to give 8.9 g (93%) of a white solid.

### Intermediate 30

### Diethyl {[(4-bromo-3-methoxyphenyl)amino]methylene}malonate

To a solution of 4-bromo-3-methoxyaniline (25 g, 0.12 mol) in CH₃CN (150 mL) was added diethylethoxymethylene malonate (27 mL, 0.13 mol). After 20 hours, the solvent was removed under reduced pressure and the residue dissolved in EtOAc. Hexane was added, and the resulting precipitate collected to give 37 g (80%) off-white solid. ¹H NMR: 10.68 (d, 1H), 8.38 (d, 1H), 7.52 (d, 1H), 7.20 (d, 1H), 6.91 (dd, 1H), 4.20 (q, 2H), 4.11 (q, 2H), 3.86 (s, 3H), 1.23 (m, 6H); m/z: 372.

### Intermediate 31

### Ethyl 7-bromo-4-chloro-6-methoxyquinoline-3-carboxylate

A mixture of ethyl 7-bromo-6-methoxy-4-oxo-1,4-dihydroquinoline-3-carboxylate

(Intermediate 32; 4.0 g, 11.6 mmol) and phosphorous oxychloride (80 mL) was heated at reflux for 2.5 hours. The solution was cooled, and poured carefully onto ice (800 g) with stirring. The mixture was carefully neutralized with 2N NaOH, and the resulting precipitate was filtered, washed with water and dried to give 3.8 g white solid. ¹H NMR (CDCl₃): 9.00 (s, 1H), 8.32 (s, 1H), 7.54 (s, 1H), 4.43 (q, 2H), 4.02 (s, 3H), 1.39 (t, 3H).

### Intermediate 32

### Ethyl 7-bromo-6-methoxy-4-oxo-1,4-dihydroquinoline-3-carboxylate

A solution of diethyl {[(3-bromo-4-methoxyphenyl)amino]methylene}malonate

(Intermediate 33; 10 g, 0.027 mol) in warm diphenyl ether (100 mL) was added dropwise over 15 minutes to refluxing diphenyl ether (100 mL). After 3 hours, the reaction mixture was cooled, and petroleum ether (120 mL) was added to the solid material, which was filtered and washed with hexane to give 8 g white solid. ¹H NMR: 8.54 (s, 1H), 7.92 (s, 1H), 7.65 (s, 1H), 4.22 (q, 2H), 3.95 (s, 3H), 1.28 (t, 3H).

### Intermediate 33

### Diethyl {[(3-bromo-4-methoxyphenyl)amino]methylene}malonate

A solution of 3-bromo-4-methoxyaniline (Intermediate 34; 8.3 g, 40.9 mmol) and diethyl ethoxymethylenemalonate (8.85 mL, 44.2 mmol) in CH₃CN (60 mL) was stirred for 2 hours. The solvent was removed under reduced pressure. Recrystallization of the residue from hexane gave 11 g white solid. ¹H NMR (CDCl₃): 10.98 (d, 1H), 8.40 (d, 1H), 7.40 (d, 1H), 7.08 (dd, 1H), 6.91 (d, 1H), 4.29 (m, 4H), 3.91 (s, 3H), 1.37 (m, 6H).

### Intermediate 34

### 3-Bromo-4-methoxyaniline

The title compound was prepared according to the procedure in Liu Y.-Y. and Munich, M., J. Label Compd Radiopharm., 18 (1981), 791-797.

### Intermediate 35

### Ethyl 6-bromo-4-chloro-7-ethoxyquinoline-3-carboxylate

To a solution of diethyl {[(4-bromo-3-ethoxyphenyl)amino]methyl}malonate (Intermediate 36; 52.9 g, 0.137 mol) in toluene (125 ml) was added POCl₃ (209.9 g, 125 mL, 1.37 mol). The reaction mixture was stirred at 110° C. for 48 hours, cooled and concentrated under reduced pressure. The residue was carefully treated with sat. NaHCO₃ solution until no more gas was evolved, and the resulting solid was filtered, washed with sat. NaHCO₃ and water, and then slurried in hot MeOH (˜200 mL), cooled and filtered to give 42 g of an orange solid.

### Intermediate 36

### Diethyl {[(4-bromo-3-ethoxyphenyl)amino]methyl}malonate

A solution of 4-bromo-3-ethoxyaniline (21 g, 0.1 mol) and diethyl ethoxymethylenemalonate (19 mL, 0.1 mol) in CH₃CN (150 mL) was stirred for 2 hours and then heated to 75° C. for 16 hours. The solvent was removed under reduced pressure and the residue recrystallized from hexane to give 25 g of white solid, which was used without further purification.

### Intermediate 37

### Ethyl 4-chloro-7-ethoxy-6-(1-methylpiperidin-4-yl)quinoline-3-carboxylate

Diethyl ({[3-ethoxy-4-(1-methylpiperidin-4-yl)phenyl]amino}methylene)malonate (Intermediate 41, 1.1 g, 2.71 mmol) in POCl₃ (15 mL) was heated to reflux for 48 hours. After cooling, the POCl₃ was removed under reduced pressure and the residue added to aqueous sodium bicarbonate (100 mL) and extracted with EtOAc (2×200 mL). The combined organic extract was dried with MgSO₄, filtered, and concentrated under reduced pressure to yield 0.81 g (80%) of the title compound, used without further purification. m/z 378.

### Intermediates 38-40

The following compounds were prepared by a similar method to Intermediate 37 using the appropriate starting materials.

| Int | Compound | M/z | SM |
|---|---|---|---|
| 38 | Ethyl 4-chloro-7-ethoxy-6-(1- | 406 | Intermediate 42 |
| | isopropylpiperidin-4- | | |
| | yl)quinoline-3-carboxylate | | |
| 39 | Ethyl 4-chloro-7-methoxy-6-(1- | 363 | Intermediate 43 |
| | methylpiperidin-4- | | |
| | yl)quinoline-3-carboxylate | | |
| 40 | Ethyl 4-chloro-6-(1-isopropylpiperidin- | 393 | Intermediate 44 |
| | 4-yl)-7-methoxyquinoline-3-carboxylate | | |

### Intermediate 41

### Diethyl ({[3-ethoxy-4-(1-methylpiperidin-4-yl)phenyl]amino}methylene)malonate

To a solution of [3-ethoxy-4-(1-methylpiperidin-4-yl)phenyl]amine hydroiodide (Intermediate 45, 1.2 g, 3.31 mmol) in acetonitrile (15 mL) was added triethylamine (0.92 mL, 6.62 mmol) and diethyl (ethoxymethylene)malonate (0.695 mL, 3.47 mmol). The reaction was stirred for 16 hours, added to aqueous sodium bicarbonate (100 mL) and extracted with EtOAc (2×200 mL). The combined organic extract was dried with MgSO₄, filtered, and concentrated under reduced pressure. The residue was purified with silica chromatography, eluting with EtOAc/hexanes (1:1) to give 1.1 g (82%) of an off white solid. m/z 406.

### Intermediates 42-44

The following compounds were prepared by a similar method to Intermediate 41 using the appropriate starting materials.

| Int | Compound | M/z | SM |
|---|---|---|---|
| 42 | Diethyl ({[3-ethoxy-4-(1-isopropylpiperidin-4- | 434 | Intermediate |
| | yl)phenyl]amino}methylene)malonate | | 46 |
| 43 | Diethyl ({[3-methoxy-4-(1-methylpiperidin-4- | 391 | Intermediate |
| | yl)phenyl]amino}methylene)malonate | | 47 |
| 44 | Diethyl ({[4-(1-isopropylpiperidin-4-yl)-3- | 419 | Intermediate |
| | methoxyphenyl]amino}methylene)malonate | | 48 |

### Intermediate 45

### [3-Ethoxy-4-(1-methylpiperidin-4-yl)phenyl]amine hydroiodide

To a 500 mL Parr bottle charged with 4-(2-ethoxy-4-nitrophenyl)-1-methylpyridinium iodide (Intermediate 51, 1.5 g, 3.88 mmol) and MeOH (100 mL) was added PtO₂ (375 mg). The vessel was placed on a Parr shaker, purged three times with H₂, and charged to 50 psi H₂. The reaction was shaken for 24 hours, then purged with nitrogen, and filtered through a bed of Celite. The filtrate was concentrated under reduced pressure to give 1.3 g (97%) of the title compound, used without further purification. m/z 235.

### Intermediates 46-48

The following compound was prepared by a similar method to Intermediate 45 using the appropriate starting materials.

| Int | Compound | M/z | SM |
|---|---|---|---|
| 46 | [3-Ethoxy-4-(1-isopropylpiperidin-4- | 263 | Intermediate 49 |
| | yl)phenyl]amine | | |
| 47 | 3-Methoxy-4-(1-methylpiperidin-4- | 221 | Intermediate 53 |
| | yl)phenyl]amine hydroiodide | | |
| 48 | [3-Methoxy-4-(1-isopropylpiperidin-4- | 249 | Intermediate 50 |
| | yl)phenyl]amine | | |

### Intermediate 49

### 4-(2-Ethoxy-4-nitrophenyl)-1-isopropyl-1,2,3,6-tetrahydropyridine

To a solution of 4-(2-ethoxy-4-nitrophenyl)-1-isopropylpyridinium iodide (Intermediate 52, 1.0 g, 2.41 mmol) in MeOH (20 mL) was added sodium borohydride (0.32 g, 8.44 mmol) in portions. The reaction was stirred for 1 hour, and acetone (5 mL) was added to destroy excess sodium borohydride. The solvents were removed under reduced pressure, and the residue dissolved in EtOAc (50 mL), added to aqueous sodium bicarbonate (100 mL), and extracted with EtOAc (2×200 mL). The combined organic extract was dried with MgSO₄, filtered, and concentrated under reduced pressure to give (0.64 g, 91%) of the title compound, used without further purification. m/z 291.

### Intermediate 50

The following compound was prepared by a similar method to Intermediate 49 using the appropriate starting materials.

| Int | Compound | M/z | SM |
|---|---|---|---|
| 50 | 4-(2-Methoxy-4-nitrophenyl)-1-isopropyl- | 277 | Intermediate 54 |
| | 1,2,3,6-tetrahydropyridine | | |

### Intermediate 51

### 4-(2-Ethoxy-4-nitrophenyl)-1-methylpyridinium iodide

To a solution of 4-(2-ethoxy-4-nitrophenyl)pyridine (Intermediate 55, 1.0 g, 4.09 mmol) in acetonitrile (20 mL) was added methyl iodide (1.16 g, 8.18 mmol). The reaction was stirred at 40° C. for 12 hours, then cooled and diluted with diethyl ether (200 mL). The resulting precipitate was filtered, washed with additional diethyl ether (100 mL), and dried to give 1.65 g (99%) of the title compound, used without further purification. m/z 260.

### Intermediates 52-54

The following compounds were prepared by a similar method to Intermediate 51 using the appropriate starting materials.

| Int | Compound | M/z | SM |
|---|---|---|---|
| 52 | 4-(2-Ethoxy-4-nitrophenyl)-1- | 288 | Intermediate 55 |
| | isopropylpyridinium iodide | | |
| 53 | 4-(2-Methoxy-4-nitrophenyl)-1- | 245 | Intermediate 56 |
| | methylpyridinium iodide | | |
| 54 | 4-(2-Methoxy-4-nitrophenyl)-1- | | Intermediate 56 |
| | isopropylpyridinium iodide | | |

### Intermediate 55

### 4-(2-Ethoxy-4-nitrophenyl)pyridine

A mixture of 1-bromo-2-ethoxy-4-nitrobenzene (5.0 g, 20.32 mmol), pyridin-4-ylboronic acid (2.50 g, 20.32 mmol), potassium carbonate (8.4 g, 60.96 mmol), and Pd(Ph₃)₄ (4.0 g, 5.08 mmol) in dioxane (60 mL) and water (6 mL) was heated to 90° C. for 24 hours under an atmosphere of argon. The reaction was cooled, diluted with water (100 mL) and extracted with EtOAc (2×200 mL). The combined organic extract was dried with MgSO₄, filtered, and concentrated under reduced pressure. The residue was purified with silica chromatography, eluting with EtOAc/hexanes (1:1) to give 3.2 g (65%) of an off white solid. m/z 245.

### Intermediate 56

The following compound was prepared by a similar method to Intermediate 55 using the appropriate starting materials.

| Int | Compound | M/z | SM |
|---|---|---|---|
| 56 | 4-(2-Methoxy-4-nitrophenyl)pyridine | 231 | 1-bromo-2-methoxy- |
| | | | 4-nitrobenzene |

### Intermediate 57

### Ethyl 6-(3-{[tert-butyl(dimethyl)silyl]oxy}propyl)-4-[(2,4-difluorophenyl)amino]-7-methoxyquinoline-3-carboxylate

A solution of {3-[(1s,5s)-9-borabicyclo[3.3.1]non-9-yl]propoxy}(tert-butyl) dimethylsilane (0.55 mmol) in THF (˜5 mL) was prepared according to the procedure of Suzuki et al (JACS, 1989, 111, 314-321). To this solution under N₂ was added K₂CO₃ (69 mg, 0.5 mmol), DMF (3 mL), [1,1′-bis(diphenylphosphino)ferrocene]-dichloropalladium(II) (28 mg, 0.34 mmol) and ethyl 6-bromo-4-[(2,4-difluorophenyl)amino]-7-methoxyquinoline-3-carboxylate (Intermediate 3; 110 mg, 0.25 mmol). After 4 hours at 50° C., another aliquot of Pd catalyst (14 mg) and K₂CO₃ (69 mg) was added, and after an additional 16 hours the reaction mixture was cooled, poured into brine (200 mL) and extracted with EtOAc (3×40 mL). The combined organic extracts were dried (Na₂SO₄), filtered, and concentrated under reduced pressure. The residue was purified by column chromatography (hexane:EtOAc) to give 100 mg of an oil. m/z: 531.

### Intermediates 58-67

The following compounds were prepared by a similar method to Intermediate 57.

| Int | Compound | M/z | SM |
|---|---|---|---|
| 58 | Ethyl 6-(3-{[tert-butyl(dimethyl)silyl]oxy}propyl)-4-[(3,4- | | Intermediate 2 |
| | dichlorophenyl)amino]-7-methoxyquinoline-3-carboxylate | | |
| 59 | Ethyl 6-(3-{[tert-butyl(dimethyl)silyl]oxy}propyl)-4-[(2,4- | | Intermediate 4 |
| | difluorophenyl)amino]-7-ethoxyquinoline-3-carboxylate | | |
| 60 | Ethyl 4-chloro-7-ethoxy-6-[3-(tetrahydro-2H-pyran-2- | 422 | Intermediate |
| | yloxy)propyl]quinoline-3-carboxylate | | 35 |
| 61 | Ethyl 7-ethoxy-4-[(4-ethylphenyl)amino]-6-[3-(tetrahydro-2H- | 507 | Intermediate 6 |
| | pyran-2-yloxy)propyl]quinoline-3-carboxylate | | |
| 62 | Ethyl 4-[(3,4-dichlorophenyl)amino]-7-ethoxy-6-[3-(tetrahydro-2H- | 547 | Intermediate 7 |
| | pyran-2-yloxy)propyl]quinoline-3-carboxylate | | |
| 63 | Ethyl 4-[(2,3-dichlorophenyl)amino]-7-ethoxy-6-[3-(tetrahydro-2H- | 547 | Intermediate 8 |
| | pyran-2-yloxy)propyl]quinoline-3-carboxylate | | |
| 64 | Ethyl 4-[(3-chloro-4-fluorophenyl)amino]-7-ethoxy-6-[3- | 531 | Intermediate 9 |
| | (tetrahydro-2H-pyran-2-yloxy)propyl]quinoline-3-carboxylate | | |
| 65 | Ethyl 6-(3-{[tert-butyl(dimethyl)silyl]oxy}propyl)-4-[(2,3- | 564 | Intermediate |
| | dichlorophenyl)amino]-7-methoxyquinoline-3-carboxylate | | 10 |
| 66 | Ethyl 6-(3-{[tert-butyl(dimethyl)silyl]oxy}propyl)-4-[(3-chloro-4- | ¹ | Intermediate |
| | fluorophenyl)amino]-7-methoxyquinoline-3-carboxylate | | 11 |
| 67 | Ethyl 6-(3-{[tert-butyl(dimethyl)silyl]oxy}propyl)-4-[(3-chloro-2- | 547 | Intermediate |
| | fluorophenyl)amino]-7-methoxyquinoline-3-carboxylate | | 12 |

¹¹H NMR: 9.57 (s, 1H), 8.94 (s, 1H), 7.62 (s, 1H), 7.23-7.36 (m, 3H), 6.98 (m, 1H), 4.18 (q, 2H), 3.97 (s, 3H), 3.55 (t, 2H), 2.65 (t, 2H), 1.62 (m, 2H), 1.25 (t, 3H), 0.85 (s, 9H), 0.00 (s, 6H)

### Intermediate 68

### Ethyl 6-(3-{[tert-butyl(dimethyl)silyl]oxy}prop-1-yn-1-yl)-4-[(4-ethylphenyl)amino]-7-methoxyquinoline-3-carboxylate

A solution of ethyl 6-bromo-4-[(4-ethylphenyl)amino]-7-methoxyquinoline-3-carboxylate (Intermediate 5; 1.0 g, 2.33 mmol), triethylamine (11.6 mL), palladium-tetrakis (triphenylphosphine) (0.269 g, 0.233 mmol), and tert-butyldimethyl(2-propynyloxy)silane (0.94 mL, 4.65 mmol) was heated at 60° C. for 24 hours. A further portion of tert-butyldimethyl(2-propynyloxy)silane (1 mL) was added and heating continued for 48 hours. After cooling, EtOAc (20 mL) and water (60 mL) was added, the aqueous layer was extracted with EtOAc and the combined organic extracts were concentrated onto silica and purified by column chromatography (Hexanes/EtOAc) to give 0.72 g (60%) of a yellow solid. ¹H NMR: 9.88 (s, 1H), 8.90 (s, 1H), 7.95 (s, 1H), 7.34 (s, 1H), 7.16 (d, 2H), 7.00 (d, 2H), 4.49 (s, 2H), 4.10 (q, 2H), 3.94 (s, 3H), 2.60 (m, 2H), 1.15 (t, 6H), 0.85 (s, 9H), −0.05 (s, 6H).

### Intermediate 69

### Ethyl 4-[(3-chloro-2-fluorophenyl)amino]-7-ethoxy-6-(3-hydroxypropyl)quinoline-3-carboxylate

A mixture of ethyl 4-chloro-7-ethoxy-6-[3-(tetrahydro-2H-pyran-2-yloxy)propyl]quinoline-3-carboxylate (Intermediate 60; 600 mg, 1.42 mmol) and 3-chloro-2-fluoroaniline (156 μL, 1.42 mmol) in EtOH (30 mL) was heated at reflux for 2 hours. The solvent was removed under reduced pressure and the residue partitioned between 0.5N NaOH (100 mL) and EtOAc (100 mL). The aqueous phase was re-extracted with EtOAc (2×100 mL) and the combined organic extracts were dried (Na₂SO₄), filtered and concentrated under reduced pressure, and the residue purified by column chromatography (Hexanes/EtOAc) to give 134 mg of an off-white solid. ¹H NMR: 9.56 (s, 1H), 8.91 (s, 1H), 7.72 (s, 1H), 7.33 (s, 1H), 7.26 (t, 1H), 7.09 (t, 1H), 6.94 (t, 1H), 4.43 (t, 1H), 4.23 (q, 2H), 4.11 (q, 2H), 3.37 (m, 2H), 2.61 (m, 2H), 1.61 (m, 2H), 1.42 (t, 3H), 1.21 (t, 3H); m/z: 447.

### Intermediates 70-78

The following compounds were prepared by a similar method to Example 2 using the appropriate starting materials.

| Int | Compound | NMR/m/z | SM |
|---|---|---|---|
| 70 | 6-Bromo-4-[(3,4-dichlorophenyl)amino]-7- | 442 | Intermediate 2 |
| | methoxyquinoline-3-carboxamide | | |
| 71 | 6-Bromo-4-[(2,4-difluorophenyl)amino]-7- | | Intermediate 3 |
| | methoxyquinoline-3-carboxamide | | |
| 72 | 7-Bromo-4-[(2,4-difluorophenyl)amino]-6- | | Intermediate |
| | methoxyquinoline-3-carboxamide | | 13 |
| 73 | 7-Bromo-4-[(3,4-dichlorophenyl)amino]-6- | | Intermediate |
| | methoxyquinoline-3-carboxamide | | 14 |
| 74 | 6-Bromo-4-[(2,4-difluorophenyl)amino]-7- | 10.60 (s, 1H), 8.90 (s, 1H), | Intermediate 4 |
| | ethoxyquinoline-3-carboxamide | 8.23 (s, 1H), 7.90 (s, | |
| | | 1H), 7.61 (s, 1H), | |
| | | 7.39 (m, 2H), 7.15 (m, 1H), | |
| | | 7.02 (m, 1H), 4.25 (q, 2H), | |
| | | 1.40 (t, 3H) | |
| 75 | 6-(3-{[tert- | 502 | Intermediate |
| | Butyl(dimethyl)silyl]oxy}propyl)-4-[(2,4- | | 57 |
| | difluorophenyl)amino]-7-methoxyquinoline- | | |
| | 3-carboxamide | | |
| 76 | 6-(3-{[tert- | 10.20 (s, 1H), 8.93 (s, 1H), | Intermediate |
| | Butyl(dimethyl)silyl]oxy}propyl)-4-[(3,4- | 8.19 (s, 1H), 7.65 (s, | 58 |
| | dichlorophenyl)amino]-7-methoxyquinoline- | 1H), 7.55 (s, 1H), | |
| | 3-carboxamide | 7.43 (d, 1H), 7.37 (s, 1H), | |
| | | 7.15 (d, 1H), 6.84 (dd, 1H), | |
| | | 3.98 (s, 3H), 3.55 (t, 2H), | |
| | | 2.66 (m, 2H), | |
| | | 1.65 (m, 2H), 0.85 (s, 9H), | |
| | | −0.03 (m, 6H) | |
| 77 | 6-(3-{[tert- | | Intermediate |
| | Butyl(dimethyl)silyl]oxy}propyl)-4-[(2,4- | | 59 |
| | difluorophenyl)amino]-7-ethoxyquinoline-3- | | |
| | carboxamide | | |
| 78 | 6-(3-{[tert-Butyl(dimethyl)silyl]oxy}prop-1- | 10.76 (s, 1H), 8.92 (s, 1H), | Intermediate |
| | yn-1-yl)-4-[(4-ethylphenyl)amino]-7- | 8.23 (s, 1H), 7.69 (s, | 68 |
| | methoxyquinoline-3-carboxamide | 1H), 7.63 (s, 1H), | |
| | | 7.29 (s, 1H), 7.10 (d, 2H), | |
| | | 6.89 (d, 2H), 4.44 (s, 2H), | |
| | | 3.91 (s, 3H), 2.57 (m, | |
| | | 2H), 1.13 (t, 3H), 0.81 (s, | |
| | | 9H), 0.00 (s, 6H); m/z | |
| | | 490 | |

### Intermediate 79

### Ethyl 6-(1-acetylpiperidin-4-yl)-4-[(2,4-difluorophenyl)amino]-7-methoxyquinoline-3-carboxylate

A solution of ethyl 4-(2,4-difluorophenylamino)-7-methoxy-6-(piperidin-4-yl)quinoline-3-carboxylate (Intermediate 83, 0.298 g, 0.67 mmol) and acetic anhydride (0.127 mL, 1.35 mmol) in dichloromethane (5 mL) was stirred for 20 hours. The reaction mixture was concentrated under reduced pressure, and the residue purified by column chromatography (dichloromethane/EtOAc) to give 0.100 g solid. m/z: 484.

### Intermediates 80-82

The following compound was prepared by a similar method to Intermediate 79 using the appropriate starting materials.

| Int | Compound | M/z | SM |
|---|---|---|---|
| 80 | Ethyl 6-(1-acetylpiperidin-4-yl)-7-ethoxy-4-[(2-fluoro-4- | 494 | Intermediate |
| | methylphenyl)amino]quinoline-3-carboxylate | | 84 |
| 81 | Ethyl 7-ethoxy-4-[(2-fluoro-4-methylphenyl)amino]-6-[1-(3- | 538 | Intermediate |
| | methoxypropanoyl)piperidin-4-yl]quinoline-3-carboxylate | | 84 |
| 82 | Ethyl 7-ethoxy-4-[(2-fluoro-4-methylphenyl)amino]-6-{1-[(2R)- | 524 | Intermediate |
| | 2-hydroxypropanoyl]piperidin-4-yl}quinoline-3-carboxylate | | 84 |

### Intermediate 83

### Ethyl 4-[(2,4-difluorophenyl)amino]-7-methoxy-6-piperidin-4-ylquinoline-3-carboxylate

To a solution of ethyl 4-(2,4-difluorophenylamino)-7-methoxy-6-(1-methylpiperidin-4-yl)quinoline-3-carboxylate (Intermediate 24, 0.301 g, 0.66 mmol) in 1,2-dichloroethane (5 mL) was added 1-chloroethyl chloroformate (0.214 mL, 1.98 mmol) and triethylamine (0.092 mL, 0.66 mmol). The reaction mixture was stirred at 75° C. for 2 hours and concentrated under reduced pressure. MeOH (10 ml) was added, the reaction was stirred at 55° C. over 72 hours and concentrated under reduced pressure. The residue was partitioned between dichloromethane and saturated NaHCO₃ solution. The aqueous phase was re-extracted with dichloromethane and the combined organic extracts were dried (Na₂SO₄), filtered and concentrated under reduced pressure to give 0.298 g brown solid, used without further purification.

### Intermediates 84-85

The following compounds were prepared by a similar method to Intermediate 83 using the appropriate starting materials.

| Int | Compound | M/z | Compound |
|---|---|---|---|
| 84 | Ethyl 7-ethoxy-4-[(2-fluoro-4- | 452 | Intermediate |
| | methylphenyl)amino]-6-piperidin- | | |
| | 4-ylquinoline-3-carboxylate | | 15 |
| 85 | Ethyl 4-[(2-fluoro-4-methylphenyl)amino]-7- | 438 | Intermediate |
| | methoxy-6-piperidin-4-ylquinoline- | | 22 |
| | 3-carboxylate | | |

### Intermediate 86

### Ethyl 6-[1-(2-{[tert-butyl(dimethyl)silyl]oxy}ethyl)piperidin-4-yl]-4-[(2-fluoro-4-methylphenyl)amino]-7-methoxyquinoline-3-carboxylate

To a solution of ethyl 4-[(2-fluoro-4-methylphenyl)amino]-7-methoxy-6-piperidin-4-ylquinoline-3-carboxylate (Intermediate 85, 0.10 g, 0.23 mmol) and (tert-butyldimethylsilyloxy)acetaldehyde (0.174 mL, 0.91 mmol) in methanol (5 mL) was added sodium triacetoxyborohydride (0.194 g, 0.91 mmol). The reaction was stirred for 24 hours, the solvent was removed under reduced pressure, and the residue purified by column chromatography (CH₂Cl₂/MeOH) to give 0.133 g yellow solid. m/z: 596.

1. A compound of formula (I): 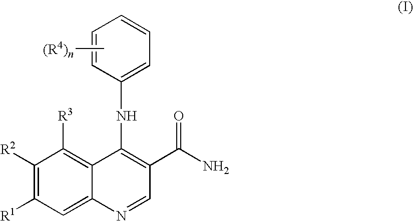
wherein:
one of R¹ and R² is selected from C₁₋₆alkyl, C₂₋₆alkenyl, C₂₋₆alkynyl, carbocyclyl or carbon-linked heterocyclyl; wherein this R¹ or R² may be optionally substituted on carbon by one or more R⁵; and wherein if said heterocyclyl contains an —NH— moiety that nitrogen may be optionally substituted by a group selected from R⁶; and
the other R¹ or R² is selected from hydrogen, halo, nitro, cyano, hydroxy, trifluoromethoxy, amino, carboxy, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, N—(C₁₋₆alkyl)-N—(C₁₋₆alkoxy)amino, C₁₋₆alkanoylamino, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl or carbon-linked heterocyclyl; wherein this R¹ or R² may be optionally substituted on carbon by one or more R⁷; and wherein if said heterocyclyl contains an —NH— moiety that nitrogen may be optionally substituted by a group selected from R⁸;
R³ is hydrogen, or halo;
R⁴ is selected from halo, nitro, cyano, hydroxy, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, N—(C₁₋₆alkyl)-N—(C₁₋₆alkoxy)amino, C₁₋₆alkanoylamino, N—(C₁₋₆alkyl)carbamoyl, N,N—(C₁₋₆alkyl)₂-carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl or heterocyclyl; wherein R⁴ may be optionally substituted on carbon by one or more R⁹; and wherein if said heterocyclyl contains an —NH-moiety that nitrogen may be optionally substituted by a group selected from R¹⁰;
or wherein if two R⁴ groups are on adjacent carbons, they may optionally form a carbocyclic ring or a heterocyclic ring; wherein said carbocyclic ring or heterocyclic ring may be optionally substituted on carbon by one or more R¹¹; and wherein if said heterocyclic ring contains an —NH— moiety that nitrogen may be optionally substituted by a group selected from R¹²;
n is 0-3; wherein the values of R⁴ are the same or different;
R⁵, R⁷, R⁹ and R¹¹ are independently selected from halo, nitro, cyano, hydroxy, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, N—(C₁₋₆alkyl)-N—(C₁₋₆alkoxy)amino, C₁₋₆alkanoylamino, N—(C₁₋₆alkyl)carbamoyl, N,N—(C₁₋₆alkyl)₂-carbamoyl, C₁₋₆alkylS(O)a wherein a is 0 to 2, C₁₋₆alkoxycarbonyl, C₁₋₆alkoxycarbonylamino, N—(C₁₋₆alkyl)sulphamoyl, N,N—(C₁₋₆alkyl)₂sulphamoyl, C₁₋₆alkylsulphonylamino, carbocyclyl-R¹³— or heterocyclyl-R¹⁴—; wherein R⁵, R⁷, R⁹ and R¹¹ independently of each other may be optionally substituted on carbon by one or more R¹⁵; and wherein if said heterocyclyl contains an —NH— moiety that nitrogen may be optionally substituted by a group selected from R¹⁶;
R¹³ and R¹⁴ are independently selected from a direct bond, —O—, —N(R¹⁷)—, —C(O)—, —N(R¹⁸)C(O)—, —C(O)N(R¹⁹)—, —S(O)ₛ—, —SO₂N(R²⁰)— or —N(R²¹)SO₂—; wherein R¹⁷, R¹⁸, R¹⁹, R²⁰ and R²¹ are independently selected from hydrogen or C₁₋₆alkyl and s is 0-2;
R⁶, R⁸, R¹⁰, R¹² and R¹⁶ are independently selected from C₁₋₆alkyl, C₁₋₆alkanoyl, C₁₋₆alkylsulphonyl, C₁₋₆alkoxycarbonyl, carbamoyl, N—(C₁₋₆alkyl)carbamoyl, N,N—(C₁₋₆alkyl)carbamoyl, benzyl, benzyloxycarbonyl, benzoyl and phenylsulphonyl; wherein R⁶, R⁸, R¹⁰, R¹² and R¹⁶ independently of each other may be optionally substituted on carbon by one or more R²²; and
R¹⁵ and R²² are independently selected from halo, nitro, cyano, hydroxy, trifluoromethoxy, trifluoromethyl, amino, carboxy, carbamoyl, mercapto, sulphamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, phenyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl or N-methyl-N-ethylsulphamoyl;
or a pharmaceutically acceptable salt thereof;
with the proviso that if R¹ is phenyl or pyrid-4-yl, R² is not hydrogen.

2. A compound of formula (I), or a pharmaceutically acceptable salt thereof, as claimed in claim 1 wherein one of R¹ and R² is selected from C₁₋₆alkyl, C₂₋₆alkynyl, carbocyclyl or carbon-linked heterocyclyl; wherein this R¹ or R² may be optionally substituted on carbon by one or more R⁵; and wherein if said heterocyclyl contains an —NH— moiety that nitrogen may be optionally substituted by a group selected from R⁶; and
the other R¹ or R² is selected from C₁₋₆alkoxy;
R⁵ is selected from hydroxy, amino, C₁₋₆alkyl, C₁₋₆alkoxy, N,N—(C₁₋₆alkyl)₂amino, C₁₋₆alkoxycarbonylamino, carbocyclyl-R¹³— or heterocyclyl-R¹⁴—;
R¹³ and R¹⁴ are independently selected from a direct bond, —O—, —N(R¹⁷)—; wherein R¹⁷ is hydrogen;
R⁶ is selected from C₁₋₆alkyl, C₁₋₆alkanoyl, C₁₋₆alkoxycarbonyl; wherein R⁶ may be optionally substituted on carbon by one or more R²²; and
R²² is selected from hydroxy or methoxy.

3. A compound of formula (I), or a pharmaceutically acceptable salt thereof, as claimed in claim 1 wherein R³ is hydrogen.

4. A compound of formula (I), or a pharmaceutically acceptable salt thereof, as claimed in claim 1 wherein R⁴ is selected from halo and C₁₋₆alkyl.

5. A compound of formula (I), or a pharmaceutically acceptable salt thereof, as claimed in claim 1 wherein n is 1 or 2; wherein the values of R⁴ are the same or different.

6. A compound of formula (I): 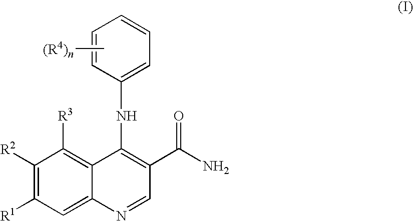
wherein:
one of R¹ and R² is selected from 1-(2-hydroxyethyl)-4-piperidyl, 1-(3-methoxypropanoyl)-4-piperidyl, 1,2,3,6-tetrahydropyridin-4-yl, 1-[(2R)-2-hydroxypropanoyl]-4-piperidyl, 1-acetyl-3,6-dihydro-2H-pyridin-4-yl, 1-acetyl-4-piperidyl, 1H-pyrazol-4-yl, 1H-pyrrol-2-yl, 1-isobutylpyrazol-4-yl, 1-isopropyl-4-piperidyl, 1-methyl-4-piperidyl, 1-tert-butoxycarbonyl-3,6-dihydro-2H-pyridin-4-yl, 3-(1-piperidyl)propyl, 3-(cyclopropylamino)propyl, 3,5-dimethylisoxazol-4-yl, 3-aminopropyl, 3-dimethylaminopropyl, 3-hydroxyprop-1-ynyl, 3-hydroxypropyl, 3-pyridyl, 4-piperidyl, 4-pyridyl, 6-methoxy-3-pyridyl, 6-oxo-1H-pyridin-3-yl, cyclopropyl, pyrimidin-5-yl, 3-(t-butoxycarbonylamino)propyl or 3-(tetrahydro-2H-pyran-2-yloxy)propyl;
the other R¹ or R² is selected from methoxy or ethoxy.
R³ is hydrogen;
R⁴ is selected from fluoro, chloro, methyl and ethyl;
n is 1 or 2; wherein the values of R⁴ are the same or different;
or a pharmaceutically acceptable salt thereof.

7. A compound of formula (I): 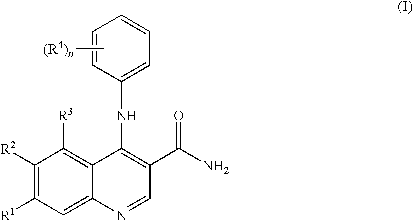
selected from:
7-ethoxy-4-[(2-fluoro-4-methylphenyl)amino]-6-(1-methylpiperidin-4-yl)quinoline-3-carboxamide;
4-[(2,4-difluorophenyl)amino]-7-ethoxy-6-(1-methyl piperidin-4-yl)quinoline-3-carboxamide;
4-[(2,4-difluorophenyl)amino]-7-ethoxy-6-(1-isopropylpiperidin-4-yl)quinoline-3-carboxamide;
7-ethoxy-4-[(2-fluoro-4-methylphenyl)amino]-6-(1-isopropylpiperidin-4-yl)quinoline-3-carboxamide;
4-[(2-fluoro-4-methylphenyl)amino]-7-methoxy-6-(1-methylpiperidin-4-yl)quinoline-3-carboxamide;
4-[(3-chloro-2-fluorophenyl)amino]-7-methoxy-6-(1-methylpiperidin-4-yl)quinoline-3-carboxamide;
4-[(2,4-difluorophenyl)amino]-7-methoxy-6-(1-methylpiperidin-4-yl)quinoline-3-carboxamide;
4-[(2-fluoro-4-methylphenyl)amino]-6-(1-isopropyl piperidin-4-yl)-7-methoxyquinoline-3-carboxamide;
4-[(2,4-difluorophenyl)amino]-6-(1-isopropylpiperidin-4-yl)-7-methoxyquinoline-3-carboxamide; and
4-[(3-chloro-2-fluorophenyl)amino]-6-(1-isopropylpiperidin-4-yl)-7-methoxyquinoline-3-carboxamide;
or a pharmaceutically acceptable salt thereof.

8. A process for preparing a compound of formula (I) or a pharmaceutically acceptable salt thereof as claimed in claim 1, which process comprises:
Process a) reacting a compound of formula (II): 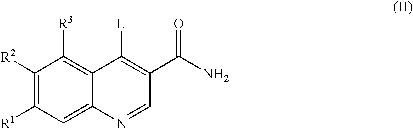
wherein L is a displaceable atom or group; with a compound of formula (III): 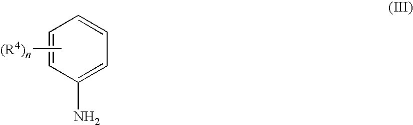
Process b) reacting a compound of formula (IV): 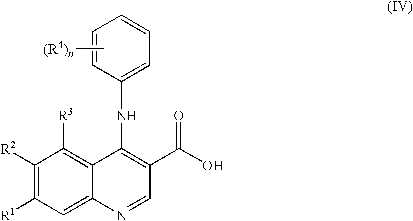
or an activated derivative thereof; with ammonia; or
Process c) reacting a compound of formula (V): 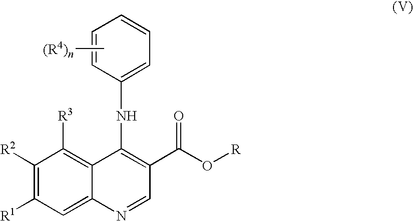
wherein R is C₁₋₆alkyl, in particular methyl and ethyl; with formamide and a base;
or
Process d) hydrolysis of a compound of formula (VI): 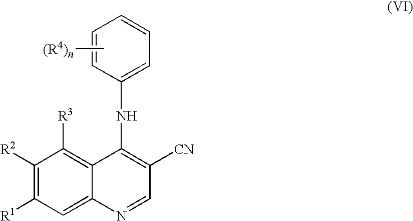
or
Process e) for compounds of formula (I) when one of R¹ and R² is selected from C₁₋₆alkyl, C₂₋₆alkenyl, C₂₋₆alkynyl, carbocyclyl or carbon-linked heterocyclyl, optionally substituted as stated herein above; by reaction of a compound of formula (VIIa) or (VIIb): 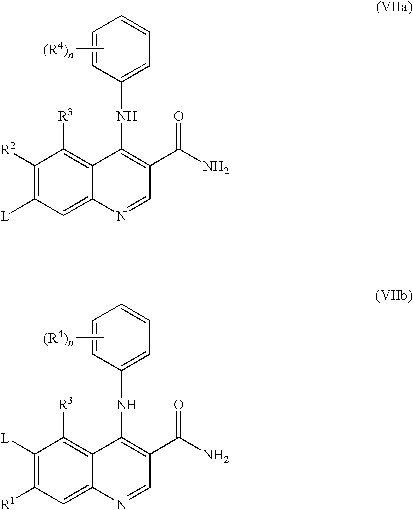
wherein L is a displaceable group; with a compound of formula (VIIIa) or (VIIIb):
R¹—B(R^{a})₂ (VIIIa)
R²—B(R^{a})₂ (VIIIb)
wherein —B(R^{a})₂ is a boronic acid derivative or trialkylborane;
and thereafter if necessary:
i) converting a compound of the formula (I) into another compound of the formula (I);
ii) removing any protecting groups;
iii) forming a pharmaceutically acceptable salt.

9. A pharmaceutical composition which comprises a compound of formula (I), or a pharmaceutically acceptable salt thereof, as claimed in claim 1, in association with a pharmaceutically-acceptable diluent or carrier.

10. A method for producing a CSF-1R kinase inhibitory effect in a warm-blooded animal in need of such treatment which comprises administering to said animal an effective amount of a compound of formula (I), or a pharmaceutically acceptable salt thereof, as claimed in claim 1.
